SEQUENCE LISTING (22 sequences)

SEQ ID NO: 1 - DNA, 21 bp, Artificial Sequence
  target sequence in MOGAT1
ccgggtcacaattatatattt

SEQ ID NO: 2 - protein, 30 aa, Artificial Sequence
  GALA Sequence
WEAALAEALAEALAEHLAEALAEALEALAA

SEQ ID NO: 3 - DNA, 23 bp, Artificial Sequence
  siRNA for MOGAT1
ccgggucacaauuauauauuutt

SEQ ID NO: 4 - DNA, 19 bp, Artificial Sequence
  luciferase target sequence for siRNA
gcgctgctggtgccaaccc

SEQ ID NO: 5 - DNA, 24 bp, Artificial Sequence
  Forward primer for MOGAT1
tgccctatcggaagctgatctaca

SEQ ID NO: 6 - DNA, 23 bp, Artificial Sequence
  Reverse primer for MOGAT1
aggtcgggttcagagtctgctga

SEQ ID NO: 7 - DNA, 23 bp, Artificial Sequence
  Forward primer for PPARa
acatttccctgtttgtggctgct

SEQ ID NO: 8 - DNA, 22 bp, Artificial Sequence
  Reverse primer for PPARa
cgtgcacaatcccctcctgcaa

SEQ ID NO: 9 - DNA, 23 bp, Artificial Sequence
  Forward primer for Cpt1a
accgccacctcttctgcctctat

SEQ ID NO: 10 - DNA, 22 bp, Artificial Sequence
  Reverse primer for Cpt1a
cgtggacaacctccatggctca

SEQ ID NO: 11 - DNA, 23 bp, Artificial Sequence
  Forward primer for Pdk4
tgcaaagatgctctgcgaccagt

SEQ ID NO: 12 - DNA, 24 bp, Artificial Sequence
  Reverse primer for Pkd4
acaatgtggattggttggcctgga

SEQ ID NO: 13 - DNA, 23 bp, Artificial Sequence
  Forward primer for Acox1
tgggccaagaagtccccactgaa

SEQ ID NO: 14 - DNA, 22 bp, Artificial Sequence
  reverse primer for Acox1
tcaaagcttcgactgcaggggc

SEQ ID NO: 15 - DNA, 19 bp, Artificial Sequence
  Forward primer for Actb
gaaggagattactgctctg

SEQ ID NO: 16 - DNA, 20 bp, Artificial Sequence
  Reverse primer for Actb
acacagagtacttgcgctca

SEQ ID NO: 17 - DNA, 20 bp, Artificial Sequence
  Forward primer for Mogat1 RT-PCR
gagtagccttgccactgata

SEQ ID NO: 18 - DNA, 19 bp, Artificial Sequence
  Reverse primer for Mogat1 RT-PCR
ataccagagtttcgtgctc

SEQ ID NO: 19 - DNA, 20 bp, Artificial Sequence
  Forward primer for Mogat2 RT-PCR
ttccagtacagctttggcct

SEQ ID NO: 20 - DNA, 20 bp, Artificial Sequence
  reverse primer for Mogat2 RT-PCR
aagtccccctaatcccacac

SEQ ID NO: 21 - DNA, 20 bp, Artificial Sequence
  Forward primer for Actb RT-PCR
acatggagaagatgtggcac

SEQ ID NO: 22 - DNA, 20 bp, Artificial Sequence
  reverse primer for Actb RT-PCR
tccatcacaatgcctgtggt

(19) United States
(12) Patent Application Publication
(10) Pub. No.: US 2013/0315925 A1
(43) Pub. Date: Nov. 28, 2013

(54) METHOD FOR TREATING AND PREVENTING TYPE 2 DIABETES

(71) Applicant: BIOMEDCORE, INC., Kanagawa (JP)

(72) Inventors: Hideyoshi HARASHIMA, Hokkaido (JP); Yasuhiro HAYASHI, Hokkaido (JP); Kazuaki KAJIMOTO, Hokkaido (JP)

(73) Assignee: BIOMEDCORE, INC., Kanagawa (JP)

(21) Appl. No.: 13/901,449

(22) Filed: May 23, 2013

Related U.S. Application Data
(60) Provisional application No. 61/651,506, filed on May 24, 2012.

(51) Int. Cl. A61K 31/7088; A61K 31/713; A61K 39/395

(52) U.S. Cl. CPC A61K 31/7088; A61K 39/3955; A61K 31/713
USPC 424/158.1; 435/14; 435/15; 514/44 R; 514/44 A; 530/389.1; 536/23.1; 536/24.5

(57) ABSTRACT

Methods and compositions for treating or preventing type 2 diabetes by inhibiting expression or activity of monoacylglycerol O-acyltransferase 1 (MOGAT1) are disclosed. Also disclosed are methods to identify such compositions.

### CROSS-REFERENCE TO RELATED APPLICATIONS

This application claims priority to U.S. provisional application No. 61/651,506, entitled “Screening of New Drugs for Type-II Diabetes,” which was filed on 24 May 2012 and is incorporated herein in its entirety by this reference. The contents of all patents, patent applications, and references cited throughout this specification are hereby incorporated by reference in their entireties.

### BACKGROUND OF THE INVENTION

Type 2 diabetes is a complex, multifactorial disease, for which genetic and environmental factors jointly determine susceptibility. Despite many efforts have been performed to screen the candidate genes by genome-wide association studies (19) and microarray studies (4), identification of new target gene is a great challenging, because the mechanisms underlying the development of type 2 diabetes have remained poorly understood. In addition, in vivo target validation methods such as generation of transgenic and knockout mice, which considered to be a gold standard, are both time-consuming and very expensive. As a treatment agent for type 2 diabetes, insulin formulations and oral antihyperglycemic drugs have been in clinical use, however more safe and effective new drug has been desired.

Insulin resistance in liver contributes greatly to the development of type 2 diabetes (1, 2). Decreased hepatic insulin sensitivity promotes hepatic glucose production and reduces glucose uptake by peripheral tissues such as skeletal muscle and adipose tissue. In addition, hepatic insulin resistance also lead to dysregulated lipid metabolism, resulting in hepatic steatosis and further systemic insulin resistance (3). Since type 2 diabetes is a complex disease, tremendous studies have been done to understand the pathogenesis of insulin resistance or type 2 diabetes by global gene expression analysis (4-6). However, it is still a very challenging task to select potential drug targets in a large amount of data.

Acyl-CoA: monoacylglycerol acyltransferase 1 (hereinafter abbreviated as “MOGAT1”) was originally identified in mice as a microsomal enzyme that catalyzes the synthesis of diacylglycerol and triacylglycerol (10). Monoacylglycerol acyltransferase (hereinafter abbreviated as “MGAT”) activity has highest in small intestine and involves with the first step in TAG re-synthesis. Monoacylglycerols produced by lipid digestive enzymes from dietary triacylglycerols in the intestinal lumen were converted into diacylglycerol and, in part, triacylglycerol though MGAT enzymes in enterocyte (12, 14). Earlier, it was reported that intestinal MGAT activities were increased in Otsuka Long Evans Tokushima Fatty (OLETF) rats (26). In addition, the protein expression of MOGAT2, a main subtype of MGAT in small intestine was upregulated and total MGAT activity was significantly enhanced in high fat diet mice (27). Furthermore, recent study demonstrated that deficient in MOGAT2 ameliorates metabolic disorders induced by high-fat feeding (15).

### SUMMARY OF THE INVENTION

The identification of disease targets and their biological validation are the first key stages in the drug discovery (7-9). Since the development of DNA microarray experiments, putative drug targets are being identified in a high-throughput manner. Once a gene is identified as a potential candidate, various strategies are utilized for target validation such as genetic studies (SNPs and genotyping), cell culture studies, and transgenic or knockout animal studies (9). Despite remarkable efforts, exploring a potential drug discovery target among candidate genes still remains a great challenge, because these strategies lack quick and direct in vivo target validation, which provides strong evidence that a particular gene is involved in the progression of disease, and delivers the highest level of target validation before work in humans. Therefore, in vivo target validation method which requires less time and disrupts arbitrary gene function, could have importance in the therapeutic target validation. In vivo siRNA delivery system that can effectively silence a specific gene is a powerful tool of predicting drug action in disease animal model. The inventors considered the combination of target discovery using DNA microarray and fast in vivo target validation facilitates a discovery of new therapeutic target.

To find a novel candidate gene involved in the pathogenesis of type 2 diabetes, we used a DNA microarray for obese diabetic KKAy or C57BL/6J mice. We reasoned that comparison of gene expression between them can't exclude some genes derived from differences in strain. Even though KK mice are the most genetically closer to KKAy mice, they are not suitable to define as normal mice because they will develop type 2 diabetes in later age. Then we decided to compare gene expression between pre- and post-diabetic KKAy mice. However, this comparison can't exclude some genes for aging, which are irresponsible for the pathogenesis of type 2 diabetes. Indeed, aging does affect hepatic lipid and glucose metabolism (20-22). To extract genes involved in the pathogenesis of type 2 diabetes, not aging, we firstly compared gene expression profiles between pre- and post-diabetic mice and then remove age-dependent genes by using differently expressed genes between 4-week and 11-week old control mice. Our data demonstrated that 129 out of 571 (22.5%) and 334 out of 821 (40.7%) were age-dependent up- and down-regulated entities in the post-diabetic stage. In particular, gene ontology analysis with down-regulated entities in the post-diabetic stage clearly showed that statistically over-represented GO terms were different between age- and diabetes-dependent entities. Furthermore, lipid metabolic process category in 442 entities includes some possible candidate genes involved with type 2 diabetes such as Cide (23, 24) and Scd1(25) (the gene expression of 11 week-old KKAy was 8.3 and 3.9 folds higher than that of 4 week-old, respectively). These results suggest that our strategy may be effective to find a disease-dependent target gene.

In this study, hepatic Mogat1, not Mogat2 expression was increased in obese diabetic mice compared with wild type mice. We also observed that Mogat1 was highly expressed in primary hepatocyte of KKAy mice (FIG. 8A). Considering the fact that hepatic MGAT activity was significantly increased in streptozotocin-induced diabetes (28), it appeared that the contribution of MOGAT1 expression is higher than that of MOGAT2. However, it has been unclear the physiological role of hepatic MGAT activities. In vivo hepatic Mogat1 silencing using siRNA loaded nanoparticle revealed that glucose and insulin response were improved, leading to decreased serum glucose level. Considering the fact that significant lowering fasting glucose level was observed, but serum insulin level was unchanged 5 days after the MOGAT1 siRNA treatment, long-term Mogat1 silencing experiment via repeated injections will be required to cure type 2 diabetes more effectively.

Interestingly, although the siRNA treatment didn't silence Mogat1 in adipose tissue, and its expression was not detected in other diabetes related organs such as skeletal muscle and intestine, systemic glucose and insulin tolerance was greatly improved. We found that silencing of Mogat1 caused ectopic fat deposition in the liver. The systemic response changes of glucose and insulin through hepatic Mogat1 silencing must be a result of the complex systemic metabolic alternations that stem from improved hepatic insulin sensitivity. One possible explanation may be caused by decreased diacylglycerol and triglyceride synthesis through hepatic Mogat1 silencing, because increased hepatic diacylglycerol content accompanied hepatic insulin resistance in obese diabetic mice (29, 30) and human (31, 32), and hepatic triacylglycerol content correlates with systemic insulin sensitivity (33, 34). The other possible explanation may be derived from improvement of nonalcoholic steatohepatitis (NASH), which characterized by fat accumulation in a context of metabolic syndrome or insulin resistance (35, 36). Indeed, we showed that some of its physiological parameters such as serum triglyceride and cholesterol levels, and ALT values, as an indicator of liver damage were mildly decreased approximately 26%, 20% and 38%, respectively. A more detailed examination will be required to understand the molecular mechanism linking NASH and insulin resistance, because insulin resistance does not always appear the progression of steatosis or NASH (37).

The knockdown of hepatic Mogat1 also increased expression of lipolysis and fatty acid oxidation enzymes. There are several reports that activation of PPAR alpha (38) or CPT1a (39) increased hepatic fatty acid oxidation and prevents insulin resistance resistance. We found that Pdk4 expression was upregulated with MOGAT1 siRNA treatment, suggesting that energy source was shifted from glucose utilization to fatty acid utilization. Although increased expression of hepatic PDK4 was observed in diabetic fatty rats (40, 41), this alternation may be a result of improvement of down-regulated lipid consumption in the liver. We believe that hepatic Mogat1 silencing caused increased fatty acid oxidation or triglyceride clearance as well as decreased diacylglycerol and triacylglycerol synthesis. However, the former effect may be less likely than the latter, since we cannot exclude the possibility that increased Ppara expression was just a result of decreased hepatic triglyceride content.

On the other hand, there are several reports about hepatic MGAT. It works as a regulatory energy source switch between triglyceride and carbohydrate in suckling rat (42), indicating that blocking hepatic MGAT pathway results in improved carbohydrate metabolism such as glucose. We are not sure what factors are involved in the regulation of hepatic Mogat1 expression, however, nuclear orphan receptor TAK1/TR4 would be one of the candidates, since it regulates the expression of several genes including Mogat1 which are involved in triglyceride accumulation (43). In addition, beraprost sodium, a prostaglandin I₂ (PGI₂) analog, ameliorated liver ectopic fat deposition in OLETF rats via decreased hepatic expression of HMG-CoA reductase (Hmgcr) and Mogat1 (44), suggesting that its target molecule or signal pathway will be a clue to discover a regulatory molecules of Mogat1 expression.

In conclusion, we have identified a hepatic Mogat1 gene involved in the pathogenesis of type 2 diabetes. Liver specific Mogat1 silencing study with siRNA loaded nanoparticle demonstrated improved systemic glucose and insulin tolerance. The potential molecular mechanism of hepatic Mogat1 silencing effect is summarized in FIG. 6. Hepatic Mogat1 silencing caused improved systemic insulin resistance through reduction of synthesis of diacylglycerol and triacylglycerol content. Decreased ectopic fat ameliorates liver toxicity and high serum cholesterol and triglycerol. In addition, fatty acid oxidation activates through PPAR alpha. Our findings suggest that hepatic MOGAT1 is a new target to combat type 2 diabetes and obesity. Moreover, our combination strategy of target identification and the following direct in vivo validation with siRNA loaded nanoparticles represents a promising technique to discover highly potential drug targets based on in vivo physiological information.

In this study, we extracted candidate genes involved in type 2 diabetes by comparing whole gene expression profiles between pre- and post-diabetic and normal mice. To examine a role of potential candidate gene in obese diabetic mice, we used a liver specific siRNA delivery system, which was developed in our laboratory (Sato Y et al. Submitted.). A strategy for type 2 diabetes target identification and validation procedures with the combination of DNA microarray and in vivo siRNA delivery system is described.

Therefore, the present invention relates to a method for treating and/or preventing type 2 diabetes in a subject, comprising administering to the subject a composition comprising a compound which is able to inhibit expression or activity of monoacylglycerol O-acyltransferase 1.

More specifically, the present invention relates to following inventions:
(1) A method for treating and/or preventing type 2 diabetes in a subject, comprising administering to the subject a composition comprising a compound which is able to inhibit expression or activity of monoacylglycerol O-acyltransferase 1 in liver of the subject.
(2) The method of (1), wherein said compound is dsRNA or antisense targeted against monoacylglycerol O-acyltransferase 1.
(3) The method of (2), wherein a target sequence of said dsRNA for the mRNA of monoacylglycerol O-acyltransferase 1 is 5′-CCGGGTCACAATTATATATTT-3′ (SEQ ID NO:1).
(4) The method of (1), wherein said compound is antagonist of monoacylglycerol O-acyltransferase 1.
(5) The method of (4), wherein said antagonist is antibody or aptamer of monoacylglycerol O-acyltransferase 1.
(6) The method of any one of (1) to (5), wherein said composition comprises said compound in lipid nanoparticle formulations.
(7) The method of (6), wherein said lipid nanoparticles comprises YSK05.
(8) The method of (6) or (7), wherein the lipid nanoparticles are modified with octaarginine and/or GALA.
(9) The method of any one of (6) to (8), wherein said compound is DNA or RNA, and wherein said DNA or RNA is included in negative charged core.
(10) The method of any one of (1) to (9), wherein said composition does not inhibit monoacylglycerol O-acyltransferase in diabetes related organ(s) other than liver.
(11) The method of (10), wherein said diabetes related organ(s) other than liver includes at least one organ selected from skeletal muscle, intestine and adipose tissue.
(12) The method of (10), wherein said diabetes related organs other than liver include skeletal muscle, intestine and adipose tissue.
(13) The method of any one of (1) to (12), wherein the subject is mammal.
(14) The method of (13), wherein the subject is human.
(15) The method of any one of (1) to (14), wherein the composition is administered in the blood stream.

In other embodiment, the present invention relates to a composition comprising a compound which is able to inhibit expression or activity of monoacylglycerol O-acyltransferase 1 for use in the treatment or prevention of type 2 diabetes.

More specifically, the present invention relates to following inventions:
(16) A composition comprising a compound which is able to inhibit expression or activity of monoacylglycerol O-acyltransferase 1 in liver of a subject for use in the treatment or prevention of type 2 diabetes.
(17) The composition of (16), wherein said compound is dsRNA or antisense DNA or RNA targeted against monoacylglycerol O-acyltransferase 1.
(18) The composition of (17), wherein a target sequence of said dsRNA for the mRNA of monoacylglycerol O-acyltransferase 1 is 5′-CCGGGTCACAATTATATATTT-3′ (SEQ ID NO:1).
(19) The composition of (16), wherein said compound is antagonist of monoacylglycerol O-acyltransferase 1.
(20) The composition of (19), wherein said antagonist is antibody or aptamer of monoacylglycerol O-acyltransferase 1.
(21) The composition of any one of (16) to (20), wherein said composition comprises said compound in lipid nanoparticle formulations.
(22) The composition of (21), wherein said lipid nanoparticles comprises YSK05.
(23) The composition of (21) or (22), wherein the lipid nanoparticles are modified with octaarginine and/or GALA.
(24) The composition of any one of (21) to (23), wherein said compound is DNA or RNA, and wherein said DNA or RNA is included in negative charged core.
(25) The composition of any one of (15) to (22), wherein said composition does not inhibit monoacylglycerol O-acyltransferase in diabetes related organ(s) other than liver.
(26) The composition of (25), wherein said diabetes related organ(s) other than liver includes at least one organ selected from skeletal muscle, intestine and adipose tissue.
(27) The composition of (25), wherein said diabetes related organs other than liver include skeletal muscle, intestine and adipose tissue.
(28) The composition of any one of (16) to (27), wherein the subject is mammal.
(29) The composition of (28), wherein the subject is human.
(30) The composition of any one of (16) to (29), wherein the composition is administered in the blood stream.

In other embodiment, the present invention relates to a method of screening a diabetes therapeutic agent or preventive agent which specifically inhibits monoacylglycerol O-acyltransferase 1 as described following (31) to (36):
(31) A method of screening a diabetes therapeutic agent or preventive agent which specifically inhibits monoacylglycerol O-acyltransferase 1, comprising:
(a) contacting a candidate compound to cells under existence of fatty acid CoA and monoacylglycerol, or contacting a control solution without the candidate compound to cells under existence of fatty acid CoA and monoacylglycerol as a control, for a predetermined period;
(b) extracting all lipids from the cells;
(c) determining amount of diacylglycerol and tryacylglycerol; and
(d) when the amount of diacylglycerol and tryacylglycerol are lower than control cells to which the candidate compound is not contacted, determining the candidate compound as a diabetes therapeutic agent or preventive agent.
(32) The method of (31), wherein the cells are cells which stably over express human monoacylglycerol O-acyltransferase 1.
(32-1) The method of (31) or (32), wherein fatty acid is RI-labeled and amount of diacylglycerol and tryacylglycerol are determined by using the RI-label.
(33) A method of screening a diabetes therapeutic agent or preventive agent which specifically inhibits monoacylglycerol O-acyltransferase 1, comprising:
(a) contacting a candidate compound to hepatic parenchymal cell culture under existence of monoacylglycerol, or contacting control solution without the candidate compound to hepatic parenchymal cell culture under existence of monoacylglycerol, for predetermined period;
(b) detecting an amount of formulation of lipid droplet in the cells;
(c) when the amount of formulation of lipid droplet are lower than control cells to which the candidate compound is not contacted, determining the candidate compound as a diabetes therapeutic agent or preventive agent.
(34) The method of (33), wherein the cells are cells which stably over express human monoacylglycerol O-acyltransferase 1.
(35) A method of screening a diabetes therapeutic agent or preventive agent which specifically inhibits monoacylglycerol O-acyltransferase 1, comprising:
(a) administering a candidate compound or control solution without the candidate compound to a diabetes model animal;
(b) extracting liver from the animal;
(c) detecting an amount of formulation of lipid droplet in the liver cells;
(d) when the amount of formulation of lipid droplet are lower than control model animal to which the candidate compound is not administered, determining the candidate compound as a diabetes therapeutic agent or preventive agent.
(36) A method of screening a diabetes therapeutic agent or preventive agent which specifically inhibits monoacylglycerol O-acyltransferase 1, comprising:
(a) administering a candidate compound or control solution without the candidate compound to a diabetes model animal;
(b) applying the animal at least one of tests selected from glucose-tolerance test, insulin-tolerance test and pyruvic tolerance test; and
(c) when one of following results are obtained from the tests, determining the candidate compound as a diabetes therapeutic agent or preventive agent:
in glucose-tolerance test, glucose peak level is lower than control model animal to which the candidate compound is not administered;
in insulin-tolerance test, insulin response was greatly improved compared with control model animal to which the candidate compound is not administered; and
in pyruvic tolerance test, de novo hepatic glucose production is reduced compared with control model animal to which the candidate compound is not administered

### BRIEF DESCRIPTION OF THE DRAWINGS

FIGS. 1A-D show a DNA microarray-based approach for selecting candidate genes for type 2 diabetes. (A) Experimental design. Liver tissues at pre-diabetic (4-week old) and post-diabetic (11-week old) stages were obtained from KKAy mice. C57BL/6J mice were used as normal controls. Each RNA sample was isolated from liver tissues and used in DNA microarray experiments. (B) Selection procedure for candidate genes involved in the pathogenesis of type 2 diabetes. First, differentially expressed genes (DEGs) between pre- and post-diabetic mice were extracted. Diabetes-dependent genes were then extracted by excluding age-dependent DEGs in KKAy mice. DEGs between 4- and 11-week old in C57BL/6J mice were defined as age-dependent genes. (C) Scatter plot of type 2 obese diabetic mice. Among the 41174 entities present on the entire mouse genome array, 19940 entities were considered to be expressed at least in a particular experimental condition. Of these 19940 entities, 1413 (583 and 830) were considered to be potentially diabetes-dependent and age-dependent entities in diabetic mice, while 986 (193 and 793) were considered to be age-dependent in normal mice. (D) Ven diagram showing differences in gene expression between diabetic and normal mice. Of the 583 entities which were up-regulated in the post-diabetic stage, 442 and 129 were determined to be diabetes-dependent, and age-dependent entities, respectively. Likewise, of the 830 entities that were down-regulated in post-diabetic stage, 487 and 334 were diabetes-dependent, and age-dependent entities, respectively.

FIGS. 2A-D show elevation of Mogat1 in diabetic mice. (A) Elevated expression of the Mogat1 gene in liver (n=4). Data represent the mean±SD. **P<0.01 vs. 4w C57BL/6J (One-way ANOVA followed by Dunnett's multiple comparison test). (B) Expression of Mogat1 in livers of other insulin-resistant mouse models. db/db mice were 9 weeks of age and db/black mice were used as controls (n=5). (C) Mice were fed a high fat diet for 14 weeks (DIO-057BL/6 mice) or standard chow (C57BL/6 mice) (n=5). Data represent the mean±SD. **P<0.01 vs. C57BL/6 mice. (D) Tissue expression pattern of Mogat1 and Mogat1. mRNA expressions in liver, adipose tissue, skeletal muscle and small intestine were detected by RT-PCR.

FIGS. 3A-G show liver specific gene silencing of Mogat1 results in improved glucose homeostasis in diabetic mice. KKAy mice received a single intravenous injections of liver specific nanoparticle loaded with MOGAT1 siRNA (5 mg/kg) or luc siRNA (5 mg/kg). Mogat1 mRNA level in (A) liver and (B) adipose tissue five days after siRNA treatment. **P<0.01 vs. siLuc treatment. (C) Blood glucose and (D) insulin levels of MOGAT1 siRNA treated mice were assayed after 8 hours of fasting. (E) Glucose tolerance test (1 g/kg, i.p.) (n=4-5). (F) Insulin tolerance test (2 U/kg, i.p.) (n=3-4). (G) Pyruvate tolerance test (2 g/kg, i.p.) (n=5). Data represent mean±SD. *P<0.05 vs. siLuc treatment; **P<0.01 vs. siLuc.

FIGS. 4A-E show gene silencing of Mogat1 ameliorates hepatic steatosis 5 days after the siRNA treatment. (A) Decreased ectopic fat deposition in liver. Lipid droplets in fatty liver tissue specimens were stained with BODIPY (green), and rhodamine-phalloidin (red) and Hoechst 33342 (cyan) for F-actin and nuclei, respectively. Typical confocal images are shown. (B) Quantitative analysis of fat storage in siRNA treated mice. The fluorescence intensities for BODIPY were normalized to the red fluorescence of F-actin. Data represent the mean±SD (n=12). **P<0.01 vs. siLuc treatment. Serum (C) TG, (D) cholesterol, and (E) ALT levels of mice treated with MOGAT1 siRNA. Data represent the mean±SD (n=4-5).

FIGS. 5A-D show knockdown of Mogat1 results in changes in the expression of lipolysis and fatty acid oxidation related genes. Relative mRNA levels of indicated genes in the livers of fasting mice treated with siRNAs, as measured by quantitative RT-PCR: (A) Ppara, (B) Pdk4, (C) Cpt1a, and (D) Acox1. Vertical axes represent the fold change in mRNA levels compared with siLuc treatment. The bars represent the fold change in expression of each gene relative to the mean expression in siLuc-treated controls±SD (n=4-5). *P<0.05 vs. siLuc treatment.

FIGS. 6A-B show a model illustrating the working hypothesis of improved glucose homeostasis via decreased ectopic fat deposition in obese diabetic mice. (A) In disease state, increased expression of Mogat1 may cause elevated ectopic fat disposition in the liver, leading to hepatic and systemic insulin resistance. (B) Mogat1 gene silencing induced decreased hepatic lipid content, which subsequently promotes improved insulin sensitivity in liver. Increased PPAR alpha expression results in activation of lipid metabolism.

FIGS. 7A-B show characteristics of experimental mice used in this study. (A) Fasting blood glucose and (B) body weight of 4-week old (pre-diabetic stage) and 11-week old (post-diabetic stage) KKAy mice. C57BL/6J mice were used as normal controls. Both mice were fasted overnight (13.5 h). (n=4). Data represent the mean±SD *P<0.05. **P<0.01 vs. 4w C57BL/6J (One-way ANOVA followed by Dunnett's multiple comparison test).

FIGS. 8A-B show siRNA transfection study in mouse primary hepatocyte. (A) Mogat1 expression in primary hepatocyte of 9-week old KKAy and C57BL/6J mouse. Primary hepatocytes were prepared as described previously (45). Mogat1 expressions were evaluated at Day 1 and Day 3 after cell seeding. The PCR products of Mogat1 were subjected to 10% native gel electrophoresis and visualized by EtBr staining. (B) Knockdown effect of MOGAT1 siRNA in primary hepatocytes. Isolated hepatocytes from 9-week old KKAy were transfected with the indicated concentrations of siRNA by using a commercially available HiPerFect transfection reagent (QIAGEN). Mogat1 mRNA levels normalized to Actb mRNA measured 48 hours after the siRNA transfection. Data represent the mean±SD (n=3). **P<0.01 vs. non-treatment (One-way ANOVA followed by Dunnett's multiple comparison test).

FIGS. 9A-F show liver specific gene silencing of Mogat1 results in improved glucose homeostasis in pre-diabetic mice. 4-week old KKAy mice received single intravenous injections of liver-specific nanoparticle loaded with MOGAT1 siRNA (5 mg/kg) or luc siRNA (5 mg/kg). (A) Mogat1 mRNA level in liver three days after siRNA treatment. (B) Blood glucose level, (C) serum AST and (D) ALT levels, (E) serum insulin, and (F) serum adiponectin. These assays were performed without fasting. Data represent mean±SD (n=4-5). **P<0.01 vs. non-treatment (One-way ANOVA followed by Dunnett's multiple comparison test).

FIG. 10 shows clearly MGAT 1 level of type 2 diabetes patient was higher than normal healthy subject.

### DETAILED DESCRIPTION OF THE INVENTION

### Method of Treatment and Composition Used Thereof

The present inventors found a linkage between Mogat1 (monoacylglycerol O-acyltransferase 1 and diabetes by originally designed in vivo genome expression analysis using siRNA, and further demonstrate that inhibition of Mogat1 in liver decreases blood sugar level and improves symptoms of diabetes. Based on these inventive discoveries, the present invention provides a method of treatment of diabetes comprising administering a composition comprising a compound which is able to inhibit expression or activity of monoacylglycerol O-acyltransferase 1. Also, the present invention provides the composition comprising a compound which is able to inhibit expression or activity of monoacylglycerol O-acyltransferase 1. In addition, the present invention is directed to method for screening an agent which can inhibit Mogat1 in liver parenchymal cells.

In the present invention, the composition can inhibit expression or activity of monoacylglycerol O-acyltransferase 1 in liver. Preferably, the composition does not inhibit expression or activity of monoacylglycerol O-acyltransferase 1 in organs (preferably diabetes related organ(s)) other than liver. This organ specific inhibitory activity of the composition may be produced by formulation of the composition, by expression system of the compound, or by any other mechanisms that can able to produce organ specific inhibition.

When the organ specific inhibitory activity of the composition is produced by formulation of the composition, the formulation can be lipid nanoparticle formulation which is specifically uptaken by liver cells. For example, such a lipid nanoparticle can be consisted of YSK-05, distearoylphosphatidylcholine, cholesterol, and mPEG-DMG. In order to be specifically uptaken by liver cells, a lipid nanoparticle can be modified with octaarginine. Any other modifications which enhance uptake by liver cells or which enhance expression of plasmid DNA or function of siRNA can be employed to the lipid nanoparticle. Such modification includes modification with GARA. GALA is synthetic pH-responsive amphipathic peptide consist of 30 amino acid with a glutamic acid-alanine-leucine-alanine (EALA) repeat that also contains a histidine and tryptophan residue as spectroscopic probes (Advanced Drug Delivery Reviews, 56(7), 23 April 2004: 967-985). For example, GALA can be a peptide with following amino acid sequence:

(SEQ ID NO: 2)
WEAALAEALAEALAEHLAEALAEALEALAA.

Thus, the present invention can be a method for treating and/or preventing type 2 diabetes in a subject, comprising administering to the subject a composition comprising a compound which is able to inhibit expression or activity of monoacylglycerol O-acyltransferase 1, wherein the composition inhibit expression or activity of monoacylglycerol O-acyltransferase 1 in liver. Also, the present invention can be a composition comprising a compound which is able to inhibit expression or activity of monoacylglycerol O-acyltransferase 1 for use in the treatment or prevention of type 2 diabetes, wherein the composition inhibit expression or activity of monoacylglycerol O-acyltransferase 1 in liver.

Whether a composition inhibits expression or activity of monoacylglycerol O-acyltransferase 1 in liver or not can be confirmed by administering a labeled composition (including a composition with labeled compound) to a subject and then confirming the distribution of the label in liver of the subject. Alternatively, the labeled composition may be applied on to liver cells in vitro and then uptake of the labeled composition by liver cells can be determined. Also, whether a composition does not inhibit expression or activity of monoacylglycerol O-acyltransferase 1 in organs other than liver can be confirmed by administering a labeled composition (including a composition with labeled compound) to a subject and then confirming the distribution of the label in organs other than liver of the subject. Alternatively, the labeled composition may be applied on to the cells of organs other than liver in vitro and then uptake of the labeled composition by the cells can be determined.

The compound of the present invention can be any compound which is able to inhibit expression or activity of monoacylglycerol O-acyltransferase 1. The compounds include a compound which inhibit expression of monoacylglycerol O-acyltransferase 1 such as dsRNA, antisense DNA or RNA and ribozyme, and a compound which inhibit activity of monoacylglycerol O-acyltransferase 1 such as antagonist of monoacylglycerol O-acyltransferase 1, antibody or aptamer of monoacylglycerol O-acyltransferase 1.

A “dsRNA”, refers to RNA containing double stranded RNA structure that inhibits gene expression by RNA interference (RNAi), and includes siRNA (short interfering RNA) and shRNA (short hairpin RNA). The dsRNA does not need to have a 100% homology to a target gene sequence so far as it inhibits expression of the target gene. A part of the dsRNA may be substituted with DNA for stabilization or other purpose(s). Preferably, the siRNA is double stranded RNA of 21 to 23 bases. The siRNA can be prepared by a method which is well known to those skilled in the art, for example, by chemical synthesis or as an analog of naturally occurring RNA. An shRNA is a short chain of RNA that has a hairpin turn structure. The shRNA can be prepared by a method that is well known to those skilled in the art, for example, by chemical synthesis or by introducing a DNA encoding shRNA into a cell and expressing the DNA.

Preferably, a target sequence of said dsRNA used in the present invention in the mRNA of monoacylglycerol O-acyltransferase 1 is 5′-CCGGGTCACAATTATATATTT-3′ (SEQ ID NO:1). Also, preferred siRNA consists of RNA of following sequence 5′-CCGGGUCACAAUUAUAUAUUUdTdT-3′ (SEQ ID NO:3) as sense sequence and RNA of complementary sequence to the sense sequence with addition of dTdT at its 3′ end as antisense sequence. An “antisense” refers to nucleic acid containing a sequence complementary to mRNA that encodes monoacylglycerol O-acyltransferase 1. The antisense may be consisted of DNA, RNA or both. The antisense does not need to be 100% complementary to mRNA of target monoacylglycerol O-acyltransferase 1. As long as it is able to specifically hybridize under stringent conditions (Sambrook et al. 1989), the antisense may contain non-complementary base. When the antisense is introduced into a cell, it binds to a target polynucleotide and inhibits transcription, RNA processing, translation or stability. The antisense includes, in addition to an antisense polynucleotide, polynucleotide mimetics, one containing modified back bone, and 3′ and 5′ terminal portions. Such antisense can be properly designed from monoacylglycerol O-acyltransferase 1 sequence information and produced using a method that is well known to those skilled in the art (for example, chemical synthesis).

A “ribozyme” is RNA possessing catalytic activity, and it is capable of cleaving, pasting, inserting, and transferring RNA. A structure of a ribozyme may be included hammerhead, hairpin, etc.

An “aptamer” is nucleic acids that bind to substance, such as protein. An aptamer may be RNA or DNA. The form of nucleic acids may be double stranded or single stranded. The length of an aptamer is not limited as far as it is able to specifically bind to a target molecule, and may be consisted of, for example, 10 to 200 nucleotides, preferably 10 to 100 nucleotides, more preferably 15 to 80 nucleotides, and further more preferably 15 to 50 nucleotides. An aptamer can be selected using a method that is well known to those skilled in the art. For example, SELEX (Systematic Evolution of Ligands by Exponential Enrichment) (Tuerk, C. and Gold, L., 1990, Science, 249, 505-510) may be employed.

An “antibody” includes full length of or fragment of antibody which specifically recognizes monoacylglycerol O-acyltransferase 1. The number of amino acid that is recognized by the antibody or its fragment is not particularly limited as long as the antibody can inhibit activity of monoacylglycerol O-acyltransferase 1. The number of the amino acid that an antibody or its fragment recognizes is at least one and more preferably at least three. An immunoglobulin class of the antibody is not limited, and may be either IgG, IgM, IgA, IgE, IgD, or IgY, and is preferably IgG. As used herein, “fragment of an antibody” is a part of the antibody (partial fragment) or a peptide containing a part of the antibody retaining an activity for an antigen of the antibody. A fragment of antibody may includes F(ab′)₂, Fab′, Fab, single chain Fv (hereinafter, abbreviated as “scFv”), disulfide bonded Fv (hereinafter, abbreviated as “dsFv”) or a polymer thereof, a dimerized V region (hereinafter, abbreviated as “Diabody”), or a peptide containing CDR. F(ab′)₂ is a fragment obtained by processing IgG with proteolytic enzyme pepsin as an antibody fragment of a molecular weight of about 100,000 with antigen avidity. A Fab′ is an antibody fragment produced by cleavage of disulfide bonds on hinge region of the F(ab′), and it has a molecular weight of about 50,000 and antigen avidity. An sdFv is a polypeptide in which one VH and one VL are joined with a peptide linker, and it has antigen avidity. A dsFv is a fragment having antigen avidity in which amino acid residues substituted with cystein in VH and VL are joined via a disulfide bond. A Diabody is a fragment of dimerized scFvs. The Diabody of the present invention may be monospecific or bispecific (multispecific antibody). The dimerized scFv may be identical or different. A peptide containing CDR is a peptide containing at least one CDR amino acid sequence selected from CDR1, CDR2, and CDR3 of variable region of a heavy chain and CDR1, CDR2, and CDR3 of variable region of a light chain. When the subject is human, it is preferable to use a humanized chimeric antibody, a humanized antibody or a human antibody as the antibody.

An antibody of the present invention can be produced by well known method, for example, by immunizing a nonhuman mammal or a bird with a peptide containing monoacylglycerol O-acyltransferase 1 or a part of monoacylglycerol O-acyltransferase 1, using an adjuvant(for example, a mineral oil or an aluminum precipitation and heat-killed bacterium or lipopolysaccharide, Freund's complete adjuvant, Freund's incomplete adjuvant, etc.) as necessary. A humanized chimeric antibody can be obtained by constructing DNA encoding VH and VL of a nonhuman animal-derived monoclonal antibody that binds to monoacylglycerol O-acyltransferase 1 to inhibit the function of monoacylglycerol O-acyltransferase 1, incorporating the constructed DNA into cDNA of constant region of a human-derived immunoglobulin and introducing the incorporated DNA into an expression vector, and introducing the vector into an adequate host cell to express it (Morrison, S. L. et al., Proc. Natl. Acad. Sci. USA, 81, 6851-6855, 1984).

A humanized antibody can be obtained by constructing DNA encoding V region in which an amino acid sequence that encodes CDR of VH and VL of a nonhuman animal-derived monoclonal antibody that binds to and inhibit the function of monoacylglycerol O-acyltransferase 1 is transplanted into FRs of VH and VL of a human antibody, incorporating the constructed DNA into cDNA of constant region of a human-derived immunoglobulin and introducing the incorporated DNA into an expression vector, and introducing the vector into an adequate host cell to express it (see L. Rieohmann et al., Nature, 332, 323, 1988; Kettleborough, C. A. et al., Protein Eng., 4, 773-783, 1991; Clark M., Immunol. Today., 21, 397-402, 2000).

A human antibody can be obtained by using a human antibody phage library or a human antibody producing transgenic mouse, for example (Tomizuka et al., Nature Genet., 15, 146-156 (1997)).

The subject of treatment or prevention of the present invention can be mammal, and preferably human. Specifically, the subject is human in need treatment and/or prevention of type 2 diabetes.

An administration route of a composition of the present invention is not limited as long as it exerts desired curative effect or preventive effect, and preferably intravascular administration. Specifically, it can be administered in the blood stream, for example, intravenously. An administration method of a drug of the present invention may include an intravenous administration by injection or intravenous drip infusion. The drug of the present invention may be administered by single, continuous, or intermittent administration. For example, a drug of the present invention may be continuously administered for 1 minute to 2 weeks. A drug of the present invention is preferably administered continuously for 5 minutes to 1 hour, and more preferably it is administered continuously for 5 minutes to 15 minutes.

A dosage of a composition of the present invention is not limited as long as an effective amount, an amount which desired curative effect or preventive effect is obtained, and can be properly determined in accordance with symptom, gender, age, etc. The dosage of a composition of the present invention can be determined, using, for example, the curative effect or preventive effect for type 2 diabetes as an indicator. The dosage of a curative drug or a preventive drug of the present invention is preferably 1 ng/kg to 10 mg/kg, more preferably 10 ng/kg to 1 mg/kg, further preferably 50 ng/kg to 500 microgram/kg, further more preferably 50 ng/kg to 100 microgram/kg, further more preferably 50 ng/kg to 50 microgram/kg, and most preferably 50 ng/kg to 5 microgram/kg.

The composition of the present invention also can be co-administered with other agent for treatment of diabetes including SU agents such as tolbutamide, glyclopyramide, acetohexamide, chlorpropamide, glybuzole, gliclazide, glibenclamide, glimepiride; biguanides such as metformin, buformine; agent for improvement of insulin resistance and thiazolidinedione derivatives such as pioglitazone, rosiglitazone; and insulin agent.

### Screening Method

The present invention also encompasses a screening method for determining at least one test agent effective for treatment of diabetes. The method comprises contacting a cell, a cell culture, or bulk cells with the test agent, wherein the cell, the cell culture, or the bulk cells express monoacylglycerol O-acyltransferase 1, and determining the activity or expression of the monoacylglycerol O-acyltransferase 1 to determine whether the test agent is an effective monoacylglycerol O-acyltransferase 1 inhibitor, wherein a test agent is considered to be effective in treatment of diabetes diabetes if the test agent is considered to be monoacylglycerol O-acyltransferase 1 inhibitor.

In further embodiments, the present invention provides a method comprising contacting a cell, a cell culture, or bulk cells with the test agent, wherein the cell, the cell culture, or the bulk cells express monoacylglycerol O-acyltransferase 1, and determining based on the inhibitory action of the test agent with the monoacylglycerol O-acyltransferase 1 whether the test agent is an monoacylglycerol O-acyltransferase 1 inhibitor suitable for treatment of diabetes.

In other embodiments, the present invention includes a method of screening a diabetes therapeutic agent or preventive agent which specifically inhibits monoacylglycerol O-acyltransferase 1

Various modifications will be apparent to those skilled in the art from this description and from practice of the invention. The scope is not intended to be limited to the particular forms disclosed and the invention covers all modifications, equivalents and alternatives falling within the spirit and scope of the invention as described here.

The method of screening of the present invention can be achieved by using common technique well known to the person skilled in the art. Also, materials used in the screening method can be obtained well know method or can be purchased from commercially available products. For example, the glucose-tolerance test can be performed by collecting a zero time (baseline) blood sample from a subject, orally administering a measured dose of glucose solution to the subject, collecting blood sample at intervals, and measuring glucose (blood sugar) level of the samples. The intervals and number of samples vary according to the purpose of the test. For simple diabetes screening, the most important sample is the 2 hour sample and the 0 and 2 hour samples may be the only ones collected. The insulin-tolerance test can be performed by fasting the subject, at 6 hours after beginning of fast collect blood sample and measure blood glucose level and then administer the subject with insulin by injection, at 30, 60 and 120 minutes after the injection, collect the second and the third blood sample and measure blood glucose level. The pyruvic tolerance test can be performed by fasting the subject overnight, a basal blood sample is collected, orally administering 50% glucose solution (2 mL/kg) which is washed down with 100 mL water, collecting further blood samples at 60 and 90 minutes after the administration, and determining pyruvate levels of the basal sample and further samples.

### EXAMPLES

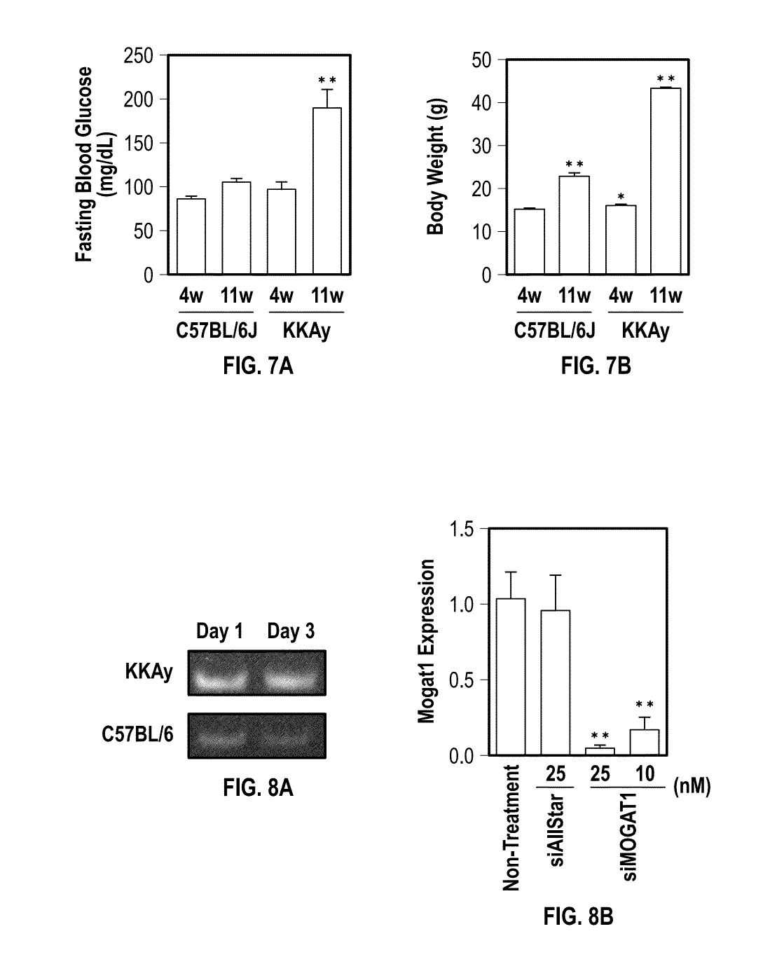

Animals. KKAy, C57BL/6J, db/db, and DIO mice were obtained from CLEA (Tokyo, Japan). Purchased KKAy mice were 4, 6 or 11 weeks old, and purchased db/db mice were 9 weeks old. All mice used in this study were males and were kept on a 12-hour light-dark cycle and fed a standard rodent chow. DIO mice were fed on a diet containing 60% fat (Research Diets, Inc.) for 10 or 14 weeks. The experimental protocols were reviewed and approved by the Hokkaido University Animal Care Committee in accordance with the guidelines for the care and use of laboratory animals. DNA microarray analysis. RNA was extracted from liver of 4-week and 11-week old KKAy and C57BL/6J mice using the RNeasy Mini Kit (QIAGEN Inc.). Fasting glucose level and body weight used in this study were shown in FIGS. 7A-B. Four RNA samples from each group were pooled into a single sample. The integrity of the pooled RNA samples was evaluated using an Agilent 2100 Bioanalyzer (Agilent Inc.). Preparation of cRNA and hybridization of whole mouse genome arrays (G4122F) were performed according to the manufacturer's instructions (Agilent Inc.). Gene expression data was analyzed by GeneSpring GX 11.5.1 software (Agilent Inc.) and an entity which was considered to be expressed in a particular experimental condition was analyzed. Results have been deposited in the Gene Expression Omnibus MIAME-compliant database (GEO, http://www.ncbi.nlm.nih.gov/geo/. Accession number: GSE1843)

RNA extraction and PCR analysis. RNA was extracted from mouse tissues (liver, epididymal fat pads, skeletal muscle, and small intestine) using an TRIzol Reagent (Invitrogen Inc.). cDNA was prepared from 1 μg of RNA using the High Capacity RNA-to-cDNA Kit (ABI), according to the manufacturer's instructions. The resulting cDNA was diluted, and a 5-μl aliquot was used in a 20-μl PCR reaction (SYBR Green; TOYOBO) containing specific primer sets (Table 2) at a concentration of 250 nM each. PCR reactions were run in duplicate and quantified using the Mx3005P Real-time QPCR system (Agilent). Cycle threshold (Ct) values were normalized to beta-actin expression, and results were expressed as a fold change of mRNA compared with control mice. Tissue distribution of Mogat1 and Mogat2 expression were determined by RT-PCR using appropriate primer sets (Table 3). The PCR products of Mogat1 and Mogat2 were subjected to gel electrophoresis (2% agarose) and visualized by EtBr staining.

Lipid nanoparticle Formulations. Liver-targeting lipid nanoparticle formulations of siRNA were prepared using the novel pH responsive cationic lipid (YSK05) (Sato Yet al. Submitted.). Lipid nanoparticle was composed of YSK-05, distearoylphosphatidylcholine, cholesterol, and mPEG-DMG, used at the molar ratio 56.2:7.0:33.8:3.0. siRNAs were formulated in lipid nanoparticles at a total lipid-to-siRNA weight ratio of approximately 5.5:1. The siRNA target sequence for the mRNA of Mogat1 was 5′-CCGGGTCACAATTATATATTT-3′ (SEQ ID NO:1), and the siRNA against luciferase mRNA was used as a control siRNA with a target sequence of 5′-GCGCTGCTGGTGCCAACCC-3′ (SEQ ID NO:4). The MOGAT1 and luciferase siRNA were obtained from Hokkaido System Science (Sapporo, Japan). The gene silencing effect of MOGAT1 siRNA was confirmed in primary hepatocyte of KKAy mice (FIGS. 8A-B). The particle size and surface charge density measurements were performed using a Zetasizer Nano ZS instrument (Worchestershire, U.K.). The mean particle size was 89±3 and 90±3 nm, and the surface charge density was 2±2 and 3±2 nm for the MOGAT1 siRNA and luc siRNA loaded lipid nanoparticles, respectively (n=5).

Lipid nanoparticle-mediated gene silencing. 10-week old KKAy mice were injected via tail vein with MOGAT1 or luc siRNA at a dose of 5 mg/kg body weight. Glucose and pyruvate tolerance test was performed on the third and fourth day after injection. Insulin tolerance test was performed on the second day after injection. Other metabolic studies and confocal imaging studies were performed on the fifth day after injection.

Metabolic Studies. Glucose- and pyruvate-tolerance tests were performed by intraperitoneal injection of glucose (1 g/kg) or pyruvate (2 g/kg) after a 6 h fast for glucose and an overnight fast for pyruvate. Insulin tolerance test was performed by intraperitoneal injection of insulin (2U/kg) 2 h after the intraperitoneal injection of glucose (2 g/kg). Blood glucose levels were measured at 0, 15, 30, 60, and 120 min after injection. Blood glucose values were determined using an Accu-Check Compact Plus (Roche Diagnostics, Indianapolis, Ind.). Serum triglyceride and cholesterol, and the serum levels of AST and ALT were measured using a colorimetric diagnostic kit (Wako Pure Chemical Industries Ltd.).

Confocal imaging studies of livers. Liver tissues were cut into thin sections (around 100 μm) using a microslicer (DSK-1000, Dosaka, Japan) and these sections were then stained with BODIPY (Invitrogen), Rhodamine-labeled phalloidin F-actin (Invitrogen) and Hoechst 33342 (Dojindo Laboratories) for an hour. After mounting the pieces on glass slides, they were viewed under a CLSM with a water immersion objective lens Plan-Apo×60/NA. Fluorescent signals were quantified using an Image-Pro® Plus-4.5 software, and fluorescence values of BODIPY, corresponding to the liver lipid droplets, were normalized to that of Rhodamine-labeled F-actin.

Statistics. All results are presented as means±SD. Statistical significance between the multiple groups was determined by ANOVA, followed by Dunnett's multiple comparison test. Significance between the two groups was calculated using student's t-test.

### Example 1

### DNA Microarray Based Target Identification for Diabetic Mice

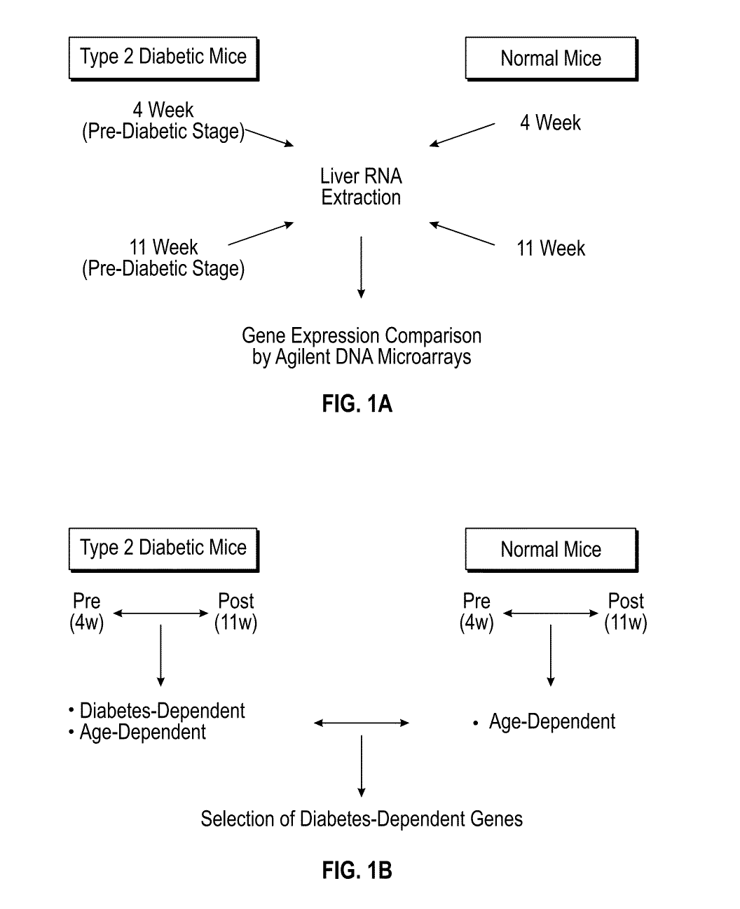

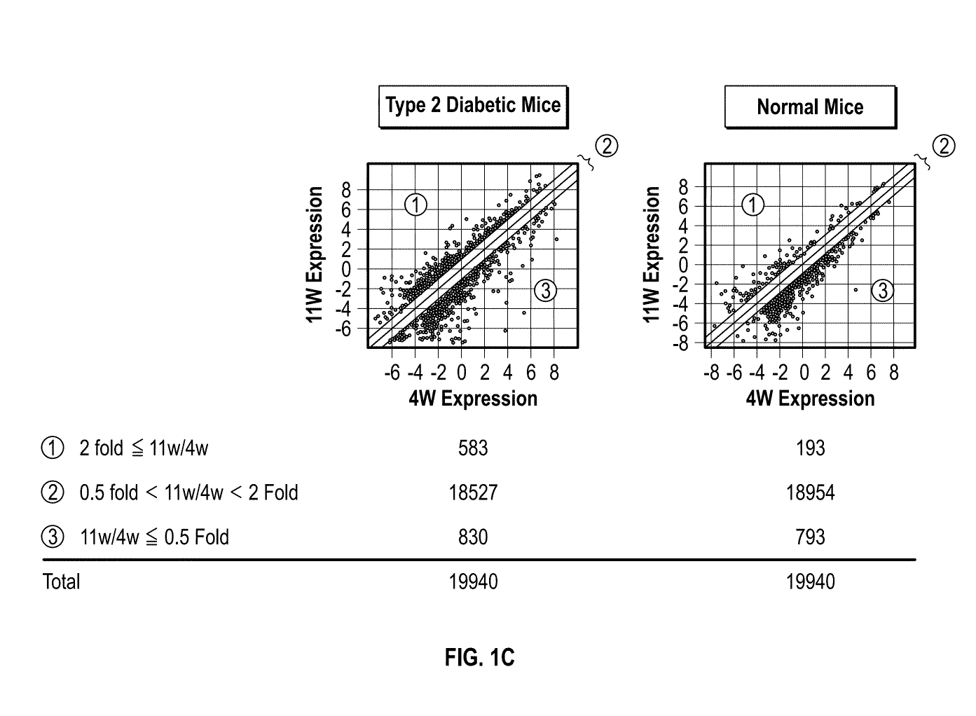

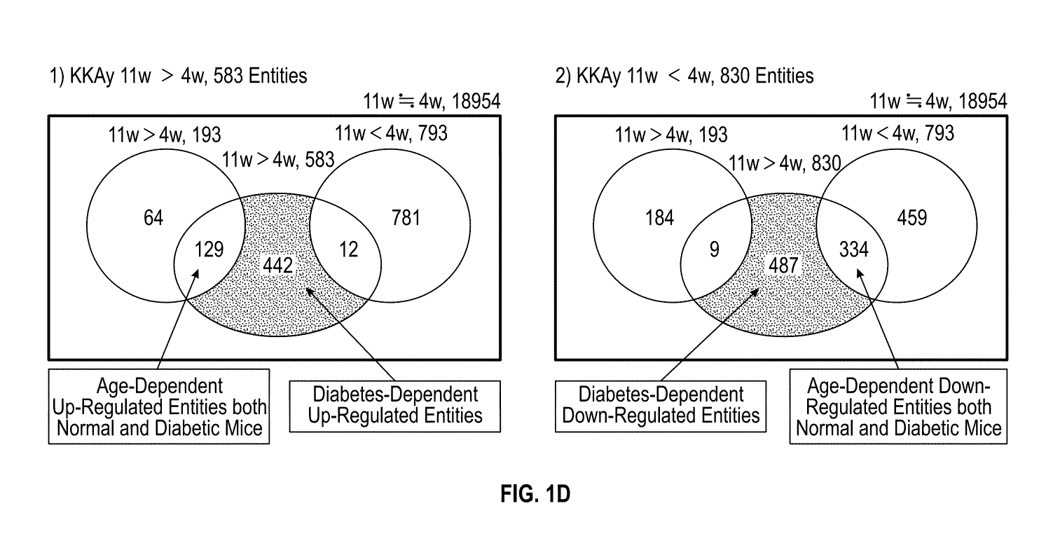

To identify candidate genes involved in the pathogenesis of KKAy mice, we performed gene expression analysis of both KKAy and C57BL/6J mice, using Agilent whole mouse genome DNA microarray (FIG. 1A). We reasoned that target genes should be differentially expressed between pre- and post-diabetic stage, however, some genes involved in aging, which are irresponsible for the pathogenesis of type 2 diabetes, might also be extracted at the same time. To efficiently identify the genes involved in the pathogenesis of type 2 diabetes, we used C57BL/6J mice as a normal control, which are most close genetic background with KKAy mice. We considered differentially expressed genes (DEGs) between 4-week and 11-week old normal mice as age-dependent genes, and then excluded these genes from DEGs between pre- and post-diabetic mice (FIG. 1B).

Scatter plot showed that 1413 (583 and 830) entities in diabetic mice were considered to be diabetes-dependent and age-dependent, and 986 (193 and 793) entities in normal were age-dependent (FIG. 1C). To distinguish diabetes- and age-dependent entities in diabetic mice, we compared DEGs of diabetic and normal mice. Among 583 entities which were up-regulated in diabetic mice, 442 and 129 were diabetes- and age-dependent, respectively. In turn, among 830 entities which were up-regulated in diabetic mice, 487 and 334 were diabetes- and age-dependent, respectively (FIG. 1D). Gene ontology analysis revealed that entities with steroid and lipid metabolic process were disproportionately represented among diabetes-dependent up-regulated entities (Table 1). On the other hand, entities with lipid metabolic process and cell cycle were over-represented among diabetes-dependent down-regulated and age-dependent entities, respectively (Table 4 and 5). A GO term; lipid metabolic process appeared in both diabetes-dependent up- and down-regulated categories. Among diabetes-dependent up-regulated entities, we focused on Mogat1 (monoacylglycerol O-acyltransferase 1) because it was reported to be involved in triglyceride synthesis and storage (10).

### Example 2

### Expression of Mogat1 in Diabetic Mice and Human

KKAy, C57BL/6J, dbdb and DIO mice were fasted overnight and livers were collected from the mice which were used to extract RNA using Trizol (Invitorogen). From the RNA, cDNA were synthesized by using High capacity RNA to DNA kit (ABI) and expression of MGAT1 gene was measured by Mx3005P Real-time QPCR (Agilent) equipment using AYBR Green agent (TOYOBO). As an internal control, expression amount of Actb was measured and expression level of MGAT1 gene was shown by relative quantitation method.

For human sample, hepatic cDNAs of health subject (C1234149) and type 2 diabetes patient (C1236149Dia) were purchased from BioChain Institute, Inc. Expression levels of MGAT1 were determined by RT-PCR method using the same manner as above described for mice.

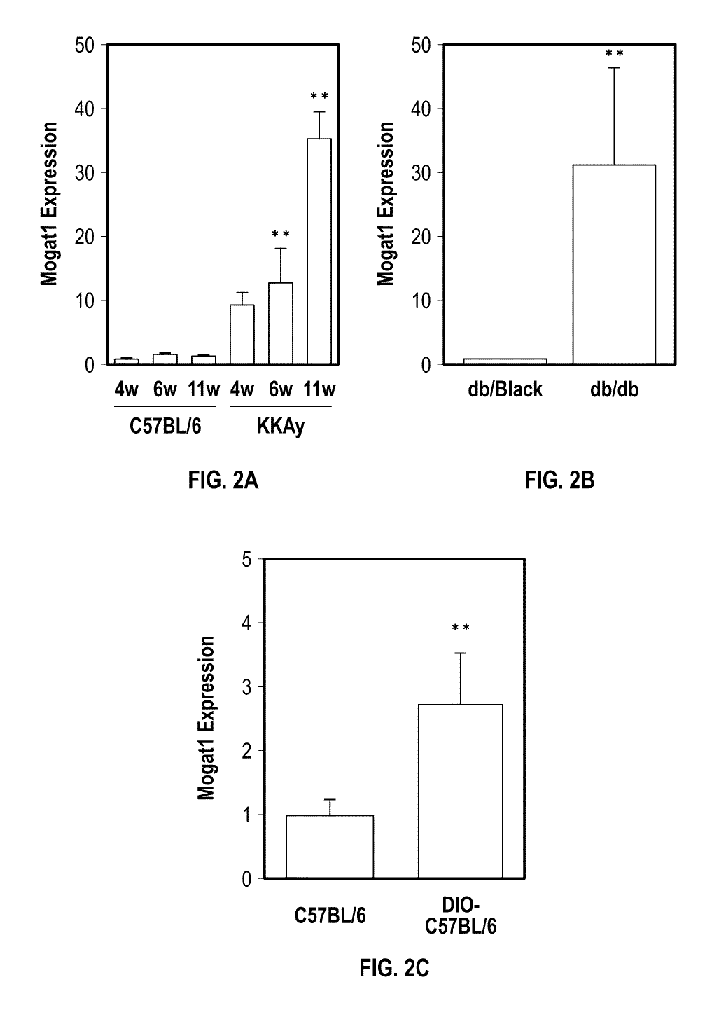

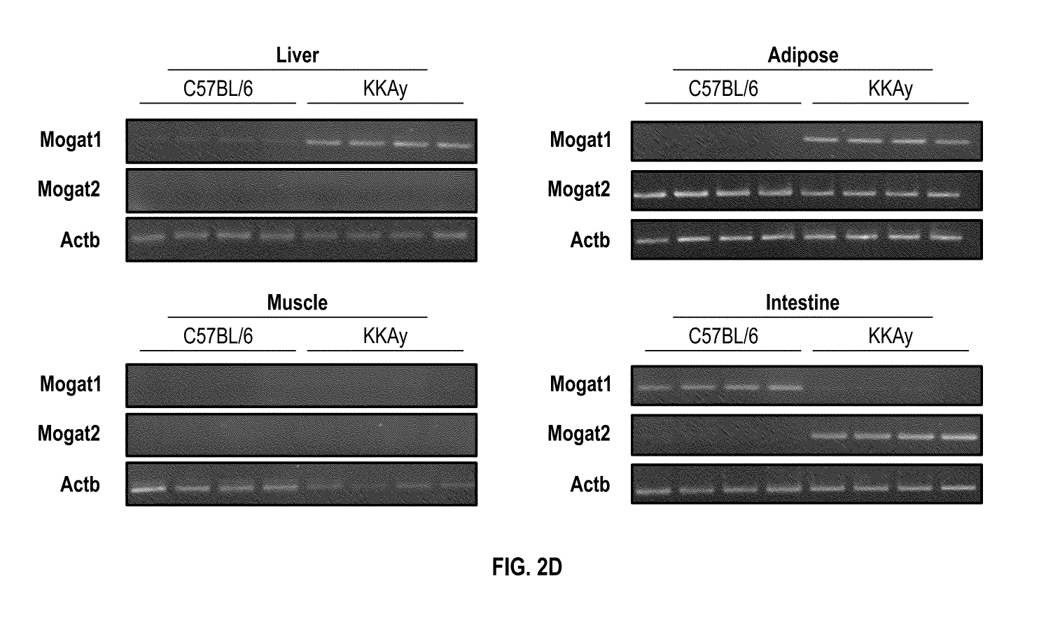

Real-time RT-PCR demonstrated that Mogat1 mRNA expression in liver was significantly higher in KKAy than C57BL/6 and the expression was gradually elevated depending on the progression of type 2 diabetes (FIG. 2A). Hepatic Mogat1 expression was also increased in two other insulin-resistant mouse models: db/db mice (32-fold increase compared with db/black mice) (FIG. 2B) and mice on a high-fat (60% fat) diet (2.7-fold increase compared with mice on a standard chow diet) (FIG. 2C). Since acyl-CoA: monoacylglycerol acyltransferase (MGAT) activity is best known for its role in fat absorption in the intestine (11, 12) and Mogat2 mRNA expression was highest in small intestine (13-15), we examined tissue distribution pattern of Mogat1 and Mogat2. Mogat1 was highly expressed in liver and adipose and not detected in skeletal muscle and intestine of KKAy mice. On the other hand, Mogat2 was highly expressed in adipose and intestine of KKAy mice (FIG. 2D).

For human samples, clearly MGAT 1 level of type 2 diabetes patient was higher than normal healthy subject (FIG. 10).

### Example 3

### Liver Specific Mogat1 Silencing Results in Improved Glucose Homeostasis in Diabetic Mice

To investigate the role of hepatic Mogat1 in vivo, liver specific siRNA delivery system was used to silence Mogat1 gene in the liver of diabetic mice. Octaarginine (R8) peptide having cell membrane permeable ability has feature to accumulate in liver (46). Multifunctional Envelope-type Nano Device (MEND) modified with R8 on its surface has been constructed as a tail vein administering carrier (48-50), which employs condensed negative charge core, reduced amount of total lipid and modification of pH responsive membrane fusion accelerating peptide (GARA) (47). Nanoparticles in which siRNA consisting of MGAT1 targeting sequence 5′-CCGGGTCACAATTATATATTT-3′ (SEQ ID NO:1) or siRNA consisting of control sequence 5′-GCGCTGCTGGTGCCAACCC-3′ (SEQ ID NO:4) (encoding targeting sequence of luciferase (luc)) was encapsulated in following lipid mixture (YSK-05; distearoylphosphatidylcholine:cholesterol:mPEG-DMG=56.2:7:33.8:3) (YSK-nanoparticle) were prepared and administered into 10 weeks old KKAy mice by tail vain injection at 5 mg/kg. Insulin-tolerance test, glucose-tolerance test (1 g/kg) and pyruvate-tolerance test (2 g/kg) were conducted two, three and four days after administration of YSK-nanoparticle, respectively. The mice were fasted for 6 hours before insulin-tolerance test (before administration of insulin) and glucose-tolerance test. Mice were fasted overnight before pyruvate-tolerance test. Blood sugar level was measured by Accu-Check Compact Plus (Roche).

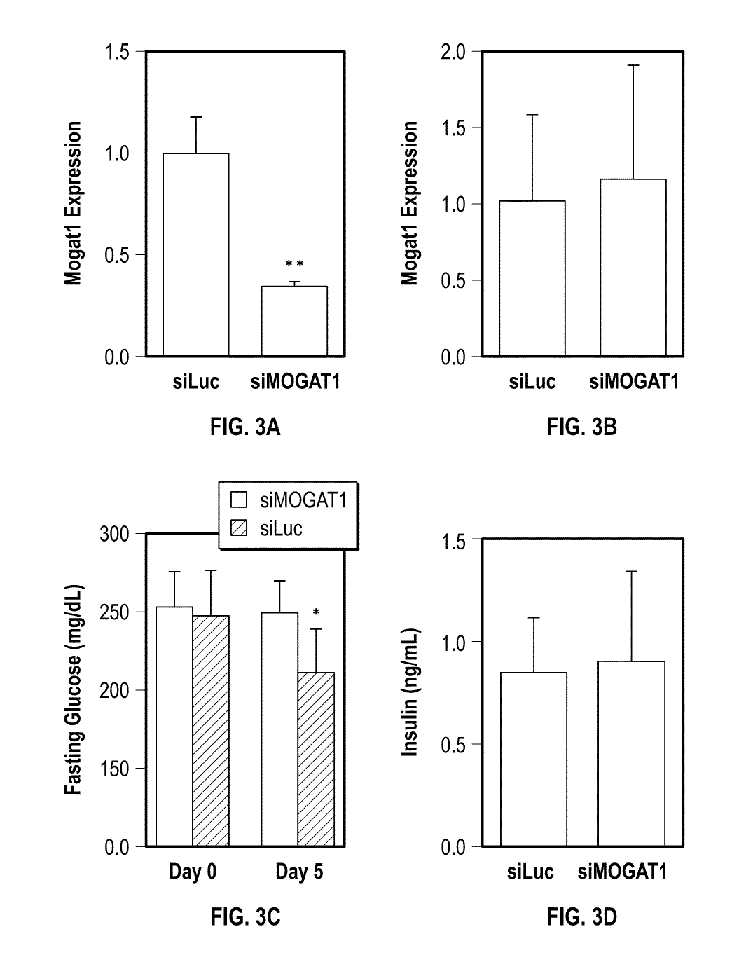

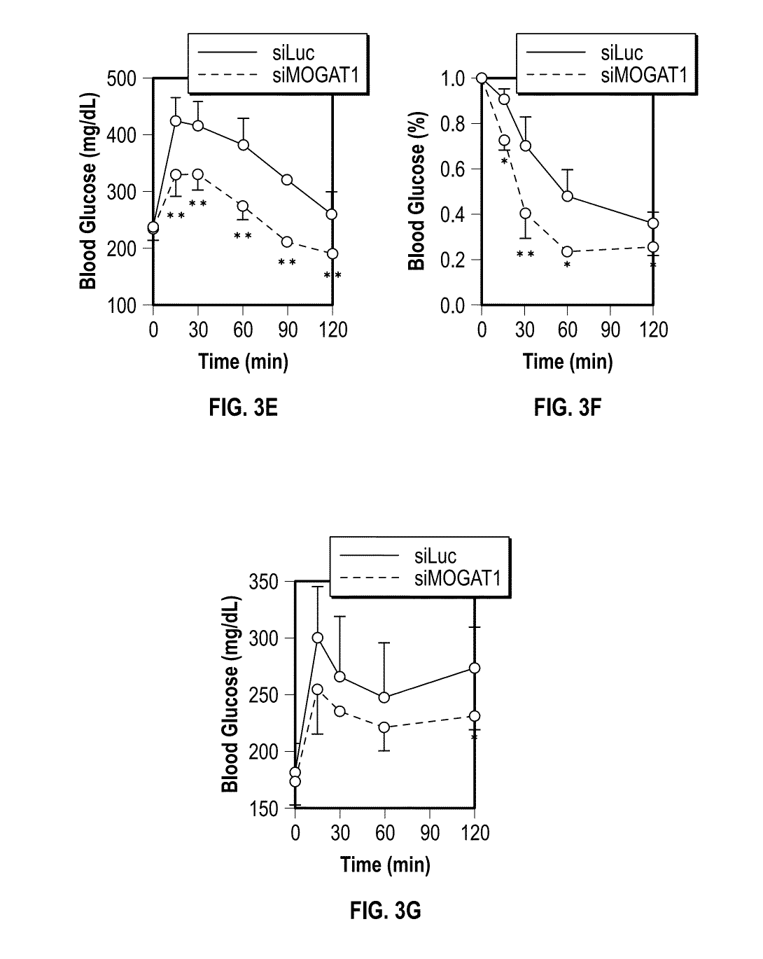

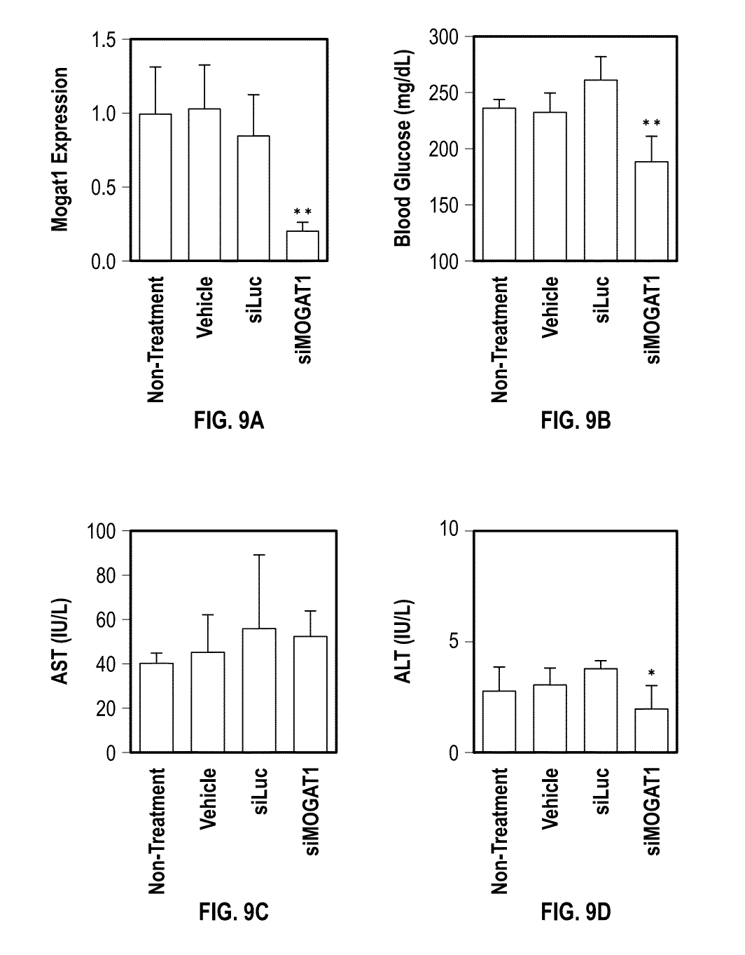

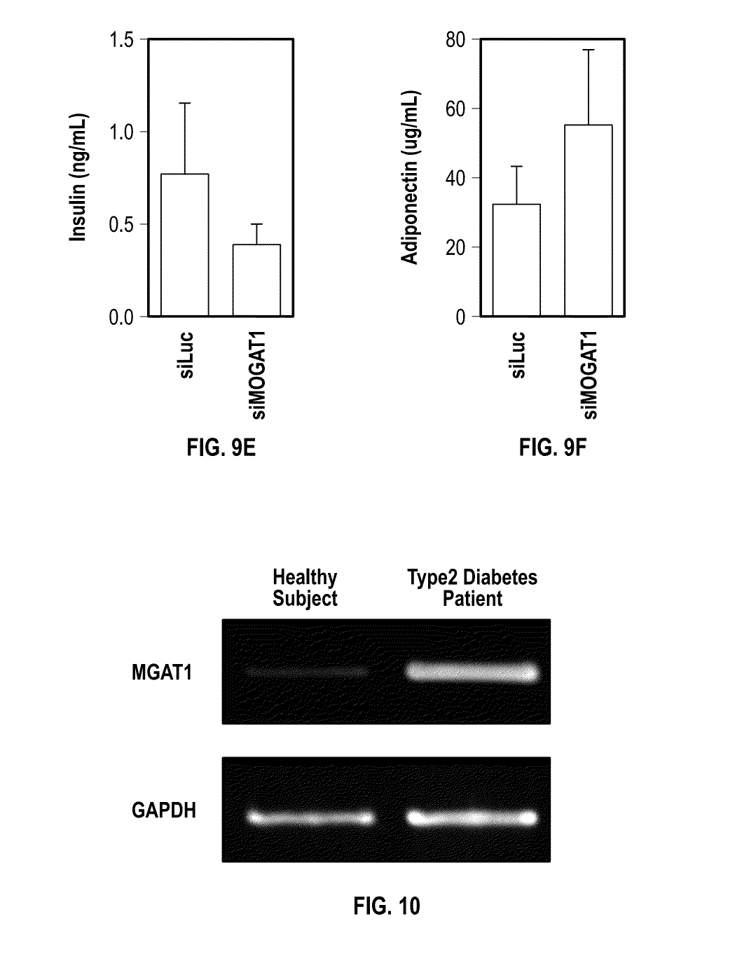

Mogat1 mRNA levels were significantly reduced by 65% in the liver (FIG. 3A) and were not reduced in the adipose tissue (FIG. 3B) 5 days after the single treatment of MOGAT1 siRNA. The siRNA delivery system didn't cause nonspecific changes in gene expression of Mogat1 and blood glucose level, nor did they cause a significant increase in aspartate aminotransferase (AST) and alanine aminotransferase (ALT) levels compared with non-treatment mice (FIGS. 9A-F). The decreased expression of Mogat1 in diabetic mice significantly lowered fasting blood glucose levels (FIG. 3C), but serum insulin levels were similar (FIG. 3D). In addition, significant reduction of blood glucose level was observed in pre-diabetic KKAy mice (FIGS. 9A-F). Glucose-tolerance test showed that elevated glucose peak level was significantly decreased by the treatment of MOGAT1 siRNA (FIG. 3E). Moreover, insulin-tolerance test demonstrated that insulin response was greatly improved in diabetic mice that were treated with MOGAT1 siRNA (FIG. 3F). A pyruvate-tolerance test revealed that de novo hepatic glucose production was mildly reduced (FIG. 3G). These data indicate that MOGAT1 is a potential therapeutic target molecule for type 2 diabetes.

### Example 4

### Reduction of Hepatic Mogat1 Expression Caused Decreased Ectopic Fat Deposition

To further explore the possible role of Mogat1 in the liver, we first examined whether silencing of Mogat1 affects triglyceride production, which is the final product of monoacylglycerol pathway (11, 13). Mice were administered YSK-nanoparticle as described in Example 3. After 5 days from the administration, mice were fasted for 8 hours and livers and bloods were collected. The collected liver was sliced into 100 μm thick slice by Microslicer (DSK-1000, Dosaka), and stained with BODIPY, Rhodamine phalloidin F-actin (invitrogen) and Hoechst 33342 (Dojindo Laboratories). The stained slice was observed by CLSM with 60×lens. Fluorescent signal was measured with Image-Pro Plus-4.5 software. From fluorescent signal of BODIPY indicating hepatic fat droplet, corresponding fluorescent signal of Rhodamine phalloidin F-actin was subtracted to conduct relative quantification. In addition, triglycerides, cholesterol, GOT and GPT levels in serum were measured by using corresponding colorimetric diagnosing kit (Wako Pure Chemical).

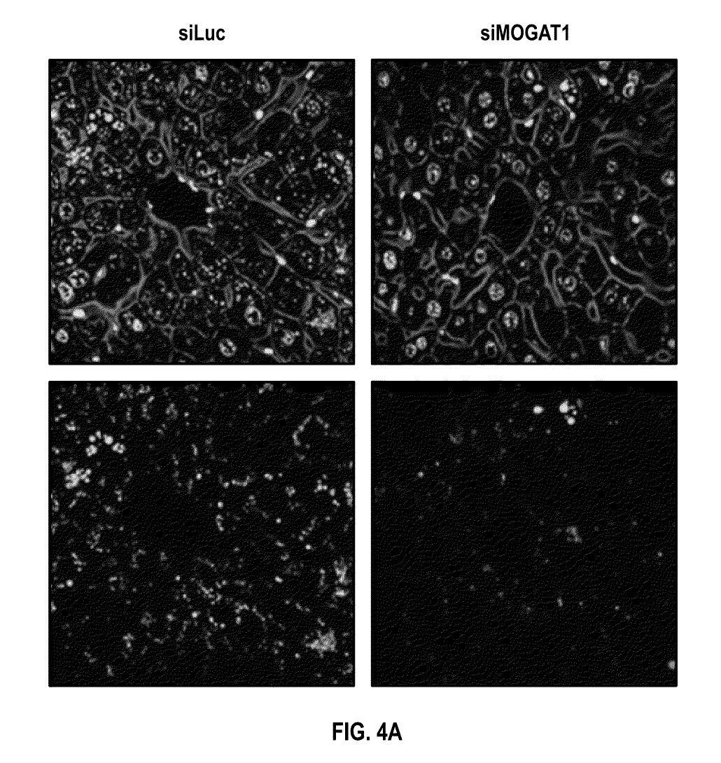

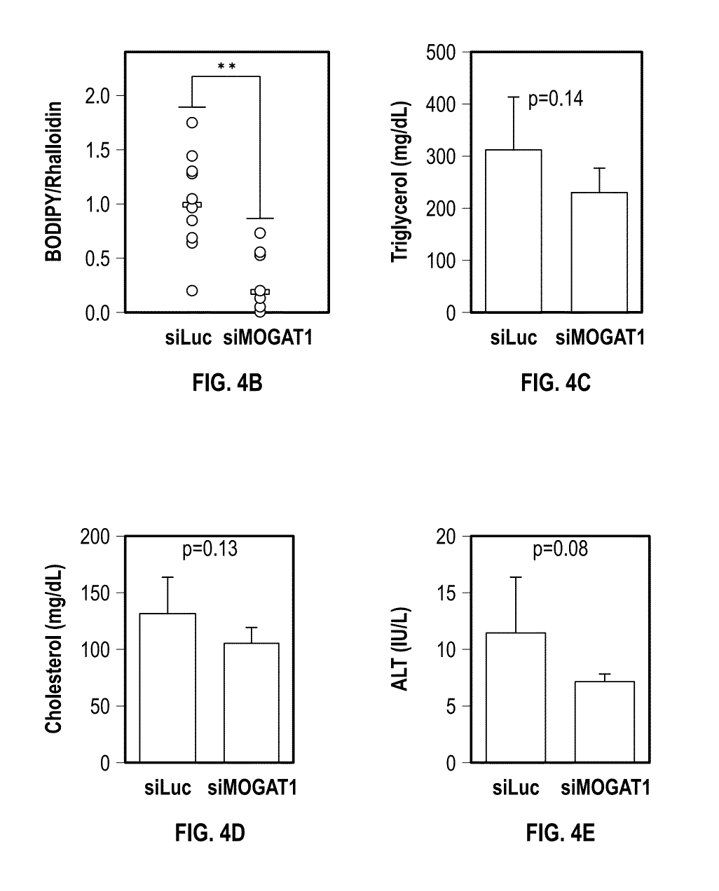

A confocal imaging study in the liver showed that mice treated with MOGAT1 siRNA showed reduction in fat storage compared with luc siRNA (FIG. 4A). Quantification of confocal images demonstrated that the amount of lipid droplets was significantly decreased (5.0-fold) (FIG. 4B), suggesting that ectopic fat deposition in liver was improved with MOGAT1 siRNA treatment. To access whether this decreased ectopic fat deposition in the liver resulted in changes in serum parameters, we analyzed serum triglyceride and cholesterol levels, which parameters were elevated in diabetic KKAy compared with C57BL/6J mice (Table 6). Mice treated with MOGAT1 siRNA showed mild decrease in serum triglyceride levels by 26% (FIG. 4C) and cholesterol levels by 20% compared with luc siRNA (FIG. 4D). In addition, ALT values, as an indicator of liver toxicity also mildly decreased (FIG. 4E), demonstrating that reduction of hepatic Mogat1 promotes decreased ectopic fat deposition, leading to improvement of fatty liver symptoms.

### Example 5

### Increased Expression of Lipolysis and Fatty Acid Oxidation Enzymes with Mogat1 Knockdown

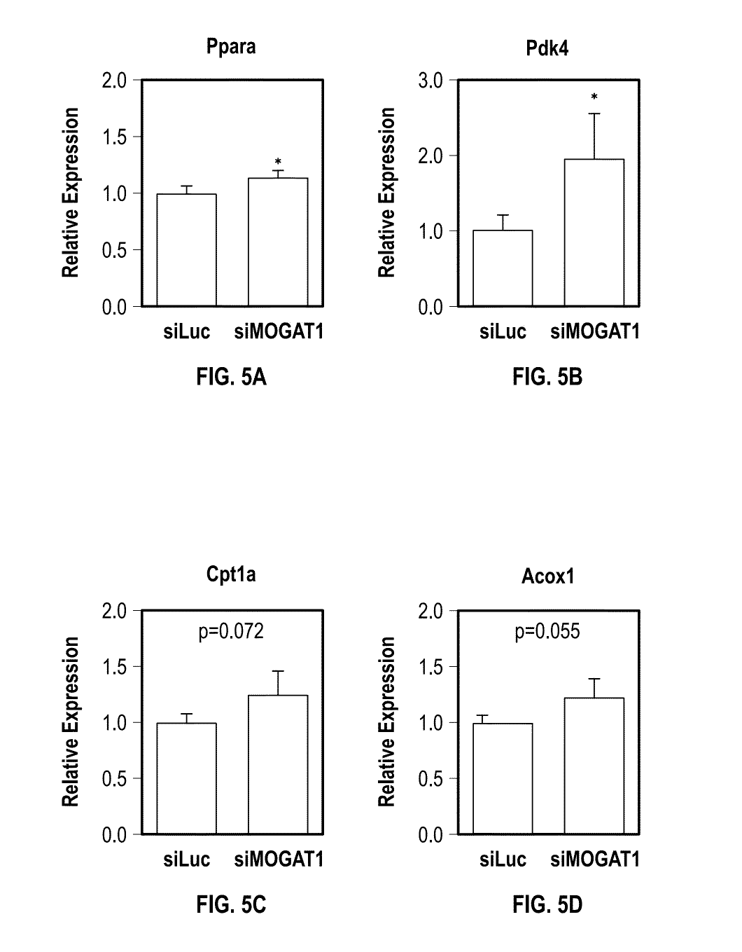

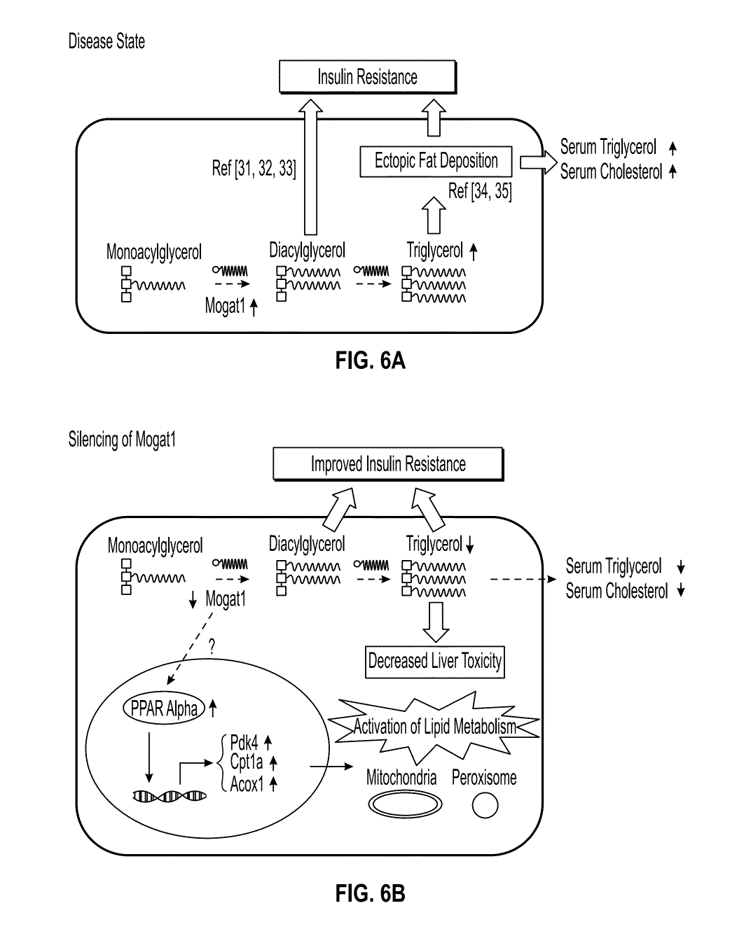

Since fatty liver disease and nonalcoholic steatohepatitis (NASH) accompany decreased PPARα expression, which regulates hepatic lipid metabolism (16-18), we reasoned that the decreased Ppara expression seen in microarray data (44% reduction compared with pre-diabetic stage) may have recovered after the treatment of MOGAT1 siRNA. Using real-time RT-PCR analysis, we measured the mRNA levels of Ppara and several key lipolysis and fatty acid oxidation related genes, pyruvate dehydrogenase kinase, isoenzyme 4 (Pdk4), carnitine palmitoyltransferase 1 (Cpt1a), and acyl-Coenzyme A oxidase 1, palmitoyl (Acox1) (FIGS. 5, A, B, C and D, respectively). Indeed, all 4 lipolysis and fatty acid oxidation genes were upregulated in livers of MOGAT1 siRNA treatment mice compared with controls.

TABLE 1
Gene Ontology analysis of diabetes-dependent up-regulated 442 entities in
diabetic mice

| GO | |
|---|---|
| ACCESSION | GO Term |
| GO: 0008202 | Steroid metabolic process |
| GO: 0006694 | Steroid biosynthetic process |
| GO: 0016126 | Sterol biosynthetic process |
| GO: 0009410 | Response to xenobiotic stimulus |
| GO: 0016614 | Oxidoreductase activity, acting on CH—OH group of donors |
| GO: 0016616 | Oxidoreductase activity, acting on the CH—OH group of |
| | donors, NAD or NADP as acceptor |
| GO: 0004089 | Carbonate dehydratase activity |
| GO: 0044444 | Cytoplasmic part |
| GO: 0005783 | Endoplasmic reticulum |
| GO: 0006629 | Lipid metabolic process |
| GO: 0006695 | Cholesterol biosynthetic process |
| GO: 0044281 | Small molecule metabolic process |
| GO: 0008610 | Lipid biosynthetic process |
| GO: 0006805 | Xenobiotic metabolic process |
| GO: 0071466 | Cellular response to xenobiotic stimulus |
| GO: 0005737 | Cytoplasm |
| GO: 0070887 | Cellular response to chemical stimulus |

TABLES 2
Primers used for real-time RT-PCR

| Primer | Sequence |
|---|---|
| Mogat1 forward | 5′-TGCCCTATCGGAAGCTGATCTACA-3′ (SEQ ID NO: 5) |
| Mogat1 reverse | 5′-AGGTCGGGTTCAGAGTCTGCTGA-3′ (SEQ ID NO: 6) |
| Ppara forward | 5′-ACATTTCCCTGTTTGTGGCTGCT-3′ (SEQ ID NO: 7) |
| Ppara reverse | 5′-CGTGCACAATCCCCTCCTGCAA-3′ (SEQ ID NO: 8) |
| Cpt1a forward | 5′-ACCGCCACCTCTTCTGCCTCTAT-3′ (SEQ ID NO: 9) |
| Cpt1a reverse | 5′-CGTGGACAACCTCCATGGCTCA-3′ (SEQ ID NO: 10) |
| Pdk4 forward | 5′-TGCAAAGATGCTCTGCGACCAGT-3′ (SEQ ID NO: 11) |
| Pdk4 reverse | 5′-ACAATGTGGATTGGTTGGCCTGGA-3′ (SEQ ID NO: 12) |
| Acox1 forward | 5′-TGGGCCAAGAAGTCCCCACTGAA-3′ (SEQ ID NO: 13) |
| Acox1 reverse | 5′-TCAAAGCTTCGACTGCAGGGGC-3′ (SEQ ID NO: 14) |
| Actb forward | 5′-GAAGGAGATTACTGCTCTGG-3′ (SEQ ID NO: 15) |
| Actb reverse | 5′-ACACAGAGTACTTGCGCTCA-3′ (SEQ ID NO: 16) |

TABLE 3
Primers used for RT-PCR

| Primer | Sequence |
|---|---|
| Mogat1 | 5′-GAGTAGCCTTGCCACTGATA-3′ |
| forward | (SEQ ID NO: 17) |
| Mogat1 | 5′-ATACCAGAGTTTCGTGCTCC-3′ |
| reverse | (SEQ ID NO: 18) |
| Mogat2 | 5′-TTCCAGTACAGCTTTGGCCT-3′ |
| forward | (SEQ ID NO: 19) |
| Mogat2 | 5′-AAGTCCCCCTAATCCCACAC-3′ |
| reverse | (SEQ ID NO: 20) |
| Actb | 5′-ACATGGAGAAGATGTGGCAC-3′ |
| forward | (SEQ ID NO: 21) |
| Actb | 5′-TCCATCACAATGCCTGTGGT-3′ |
| reverse | (SEQ ID NO: 22) |

TABLE 4
Gene Ontology analysis of diabetes-dependent down-regulated 487
entities in diabetic mice

| GO ACCESSION | GO Term |
|---|---|
| GO: 0006629 | Lipid metabolic process |
| GO: 0051338 | Regulation of transferase activity |
| GO: 0033674 | Positive regulation of kinase activity |
| GO: 0051347 | Positive regulation of transferase activity |
| GO: 0043549 | Regulation of kinase activity |
| GO: 0065008 | Regulation of biological quality |
| GO: 0044281 | Small molecule metabolic process |
| GO: 0045860 | Positive regulation of protein kinase activity |
| GO: 0043085 | Positive regulation of catalytic activity |
| GO: 0045859 | Regulation of protein kinase activity |
| GO: 0004497 | Monooxygenase activity |
| GO: 0042180 | Cellular ketone metabolic process |
| GO: 0048878 | Chemical homeostasis |
| GO: 0004062 | Aryl sulfotransferase activity |
| GO: 0051782 | Negative regulation of cell division |
| GO: 0055088 | Lipid homeostasis |
| GO: 0048015 | Phosphoinositide-mediated signaling |

TABLE 5
Gene Ontology analysis of age-dependent down-regulated 334 entities
in both diabetic and normal mice

| GO ACCESSION | GO Term |
|---|---|
| GO: 0000278 | mitotic cell cycle |
| GO: 0000087 | M phase of mitotic cell cycle |
| GO: 0007049 | cell cycle |
| GO: 0000280 | nuclear division |
| GO: 0007067 | mitosis |
| GO: 0048285 | organelle fission |
| GO: 0000279 | M phase |
| GO: 0022403 | cell cycle phase |
| GO: 0022402 | cell cycle process |
| GO: 0051301 | cell division |
| GO: 0000775 | chromosome, centromeric region |
| GO: 0005819 | spindle |
| GO: 0005694 | chromosome |
| GO: 0000793 | condensed chromosome |
| GO: 0044427 | chromosomal part |
| GO: 0016043 | cellular component organization |
| GO: 0071840 | cellular component organization or |
| | biogenesis |
| GO: 0000776 | kinetochore |

TABLE 6
Characteristics of 10-week old C57BL/6

| | C57BL/6J | KKAy |
|---|---|---|
| Body Weight (g) | 18.4 ± 0.3 | 37.9 ± 0.9** |
| serum glucose (mg/dl) | 88.8 ± 7.6 | 169.1 ± 36.7** |
| serum cholesterol (mg/dl) | 80.8 ± 4.1 | 132.0 ± 5.5** |
| serum triglyceride (mg/dl) | 107.9 ± 28.1 | 172.5 ± 20.0** |
| serum NEFA (μEq/l) | 1.7 ± 0.4 | 1.5 ± 0.1 |

Data are presented as the mean ± SD (n = 4), **P < 0.01).

### REFERENCES

[1] Kim S P, Ellmerer M, Van Citters G W, Bergman R N. Primary of hepatic insulin resistance in the development of the metabolic syndrome induced by an isocaloric moderate-fat diet in the dog. Diabetes. 2003; 52(10):2453-60.
[2] Taniguchi C M, Ueki K, Kahn R. Complementary roles of IRS-1 and IRS-2 in the hepatic regulation of metabolism. J Clin Invest. 2005; 115(3):718-27.
[3] Shimomura I, Matsuda M, Hammer RE, Bashmakov Y, Brown M S, Goldstein J L. Decreased IRS-2 and increased SREBP-1c lead to mixed insulin resistance and sensitivity in livers of lipodystrophic and ob/ob mice. Mol Cell. 2000; 6(1):77-86.
[4] Buechler C, Schaffler A. Does global gene expression analysis in type 2 diabetes provide an opportunity to identify highly promising drug targets?. Endocr Metab Immune Disord Drug Targets. 2007; 7(4):250-258.
[5] Hayashi Y, Kajimoto K, Iida S, Sato Y, Mizufune S, Kaji N, Kamiya H, Baba Y, Harashima H. DNA microarray analysis of whole blood cells and insulin-sensitive tissues reveals the usefulness of blood RNA profiling as a source of markers for predicting type 2 diabetes. Biol Pharm Bull. 2010; 33(6): 1033-1042.
[6] Keller M P, Attie A D. Physiological insights gained from gene expression analysis in obesity and diabetes. Annu Rev Nutr. 2010; 30:341-364.
[7] Wang S, Sim T B, Kim Y S, Chang Y T. Tools for target identification and validation. Curr Opin Chem Biol. 2004; 8(4):371-377.
[8] Gremel G, Rafferty M, Lau T Y, Gallagher W M. Identification and functional validation of therapeutic targets for malignant melanoma. Crit Rev Oncol Hematol. 2009; 72(3):194-214.
[9] Jayapal M, Melendez A J. DNA microarray technology for target identification and validation. Clin Exp Pharmacol Physiol. 2006; 33(5-6):496-503.
[10] Yen C L, Stone S J, Cases S, Zhou P, Farese R V Jr. Identification of a gene encoding MGAT1, a monoacylglycerol acyltransferase. Proc Natl Acad Sci USA. 2002; 99(13):8512-8517.
[11] Lehner R, Kuksis A. Biosynthesis of triacylglycerols. Prog Lipid Res. 1996; 35(2): 169-201.
[12] Shi Y, Burn P. Lipid metabolic enzymes: emerging drug targets for the treatment of obesity. Nat Rev Drug Discov. 2004; 3 (8):695-710.
[13] Cao J, Lockwood J, Burn P, Shi Y. Cloning and functional characterization of a mouse intestinal acyl-CoA:monoacylglycerol acyltransferase, MGAT2. J Biol Chem. 2003; 278(16):13860-13866.
[14] Yen C L, Farese R V Jr. MGAT2, a monoacylglycerol acyltransferase expressed in the small intestine. J Biol Chem. 2003; 278(20):18532-7.
[15] Yen C L, Cheong M L, Grueter C, Zhou P, Moriwaki J, Wong J S, Hubbard B, Marmor S, Farese R V Jr. Deficiency of the intestinal enzyme acyl CoA:monoacylglycerol acyltransferase-2 protects mice from metabolic disorders induced by high-fat feeding. Nat Med. 2009; 15(4):442-446.
[16] Yeon J E, Choi K M, Baik S H, Kim K O, Lim H J, Park K H, Kim J Y, Park J J, Kim J S, Bak Y T, Byun K S, Lee C H. Reduced expression of peroxisome proliferator-activated receptor-alpha may have an important role in the development of non-alcoholic fatty liver disease. J Gastroenterol Hepatol. 2004; 19(7):799-804.
[17] Svegliati-Baroni G, Candelaresi C, Saccomanno S, Ferretti G, Bachetti T, Marzioni M, De Minicis S, Nobili L, Salzano R, Omenetti A, Pacetti D, Sigmund S, Benedetti A, Casini A. A model of insulin resistance and nonalcoholic steatohepatitis in rats: role of peroxisome proliferator-activated receptor-alpha and n-3 polyunsaturated fatty acid treatment on liver injury. Am J Pathol. 2006; 169(3):846-860.
[18] Cong W N, Tao R Y, Tian J Y, Liu G T, Ye F. The establishment of a novel non-alcoholic steatohepatitis model accompanied with obesity and insulin resistance in mice. Life Sci. 2008; 82(19-20):983-90.
[19] McCarthy M I, Zeggini E. Genome-wide association scans for type 2 diabetes: new insights into biology and therapy. Trends Pharmacol Sci. 2007; 28(12):598-601.
[20] Barakat H A, Dohm G L, Shukla N, Marks R H, Kern M, Carpenter J W, Mazzeo R S. Influence of age and exercise training on lipid metabolism in Fischer-344 rats. J Appl Physiol. 1989; 67(4):1638-1642.
[21] Ahren B, Pacini G. Age-related reduction in glucose elimination is accompanied by reduced glucose effectiveness and increased hepatic insulin extraction in man. J Clin Endocrinol Metab. 1998; 83(9):3350-3356.
[22] Elahi D, Muller D C, Egan J M, Andres R, Veldhuist J, Meneilly G S. Glucose tolerance, glucose utilization and insulin secretion in aging. Novartis Found Symp. 2002; 242:222-242;discussion 242-246.
[23] Puri V, Ranjit S, Konda S, Nicoloro S M, Straubhaar J, Chawla A, Chouinard M, Lin C, Burkart A, Corvera S, Perugini R A, Czech M P. Cidea is associated with lipid droplets and insulin sensitivity in humans. Proc Natl Acad Sci USA. 2008; 105(22):7833-7838.
[24] Hall A M, Brunt E M, Chen Z, Viswakarma N, Reddy J K, Wolins N E, Finck B N. Dynamic and differential regulation of proteins that coat lipid droplets in fatty liver dystrophic mice. J Lipid Res. 2010; 51(3):554-563.
[25] Miyazaki M, Flowers M T, Sampath H, Chu K, Otzelberger C, Liu X, Ntambi J M. Hepatic stearoyl-CoA desaturase-1 deficiency protects mice from carbohydrate-induced adiposity and hepatic steatosis. Cell Metab. 2007; 6(6):484-96.
[26] Luan Y, Hirashima T, Man Z W, Wang M W, Kawano K, Sumida T. Pathogenesis of obesity by food restriction in OLETF rats-induced intestinal monoacylglycerol acyltransferase activities may be a crucial factor. Diabetes Res Clin Pract. 2002; 57(2):75-82.
[27] Cao J, Hawkins E, Brozinick J, Liu X, Zhang H, Burn P, Shi Y. A predominant role of acyl-CoA:monoacylglycerol acyltransferase-2 in dietary fat absorption implicated by tissue distribution, subcellular localization, and up-regulation by high fat diet. J Biol Chem. 2004; 279(18):18878-18886.
[28] Mostafa N, Bhat B G, Coleman R A. Increased hepatic monoacylglycerol acyltransferase activity in streptozotocin-induced diabetes: characterization and comparison with activities from adult and neonatal rat liver. Biochim Biophys Acta. 1993; 1169(2):189-195.
[29] Saha A K, Kurowski T G, Colca J R, Ruderman N B. Lipid abnormalities in tissues of the KKAy mouse: effects of pioglitazone on malonyl-CoA and diacylglycerol. Am J Physiol. 1994; 267(1 Pt 1):E95-101.
[30] Kraegen E W, Saha A K, Preston E, Wilks D, Hoy A J, Cooney G J, Ruderman N B. Increased malonyl-CoA and diacylglycerol content and reduced AMPK activity accompany insulin resistance induced by glucose infusion in muscle and liver of rats. Am J Physiol Endocrinol Metab. 2006; 290(3):E471-479.
[31] Jornawaz F R, Birkenfeld A L, Jurczak M J, Kanda S, Guigni B A, Jiang D C, Zhang D, Lee H Y, Samuel V T, Shulman G I. Hepatic insulin resistance in mice with hepatic overexpression of diacylglycerol acyltransferase 2. Proc Natl Acad Sci USA. 2011; 108(14):5748-5752.
[32] Kumashiro N, Erion D M, Zhang D, Kahn M, Beddow S A, Chu X, Still C D, Gerhard G S, Han X, Dziura J, Petersen K F, Samuel V T, Shulman G I. Cellular mechanism of insulin resistance in nonalcoholic fatty liver disease. Proc Natl Acad Sci USA. 2011; 108(39):16381-16385.
[33] Bays H, Mandarino L, DeFronzo R A. Role of the adipocyte, free fatty acids, and ectopic fat in pathogenesis of type 2 diabetes mellitus: peroxisomal proliferator-activated receptor agonists provide a rational therapeutic approach. J Clin Endocrinol Metab. 2004; 89(2):463-478.
[34] Lettner A, Roden M. Ectopic fat and insulin resistance. Curr Diab Rep. 2008; 8(3):185-191.
[35] Bugianesi E, Moscatiello S, Ciaravella M F, Marchesini G. Insulin resistance in nonalcoholic fatty liver disease. Curr Pharm Des. 2010; 16(17):1941-1951.
[36] Hebbard L, George J. Animal models of nonalcoholic fatty liver disease. Nat Rev Gastroenterol Hepatol. 2011; 8(1):35-44.
[37] Maeda N, Shimomura I, Kishida K, Nishizawa H, Matsuda M, Nagaretani H, Furuyama N, Kondo H, Takahashi M, Arita Y, Komuro R, Ouchi N, Kihara S, Tochino Y, Okutomi K, Horie M, Takeda S, Aoyama T, Funahashi T, Matsuzawa Y. Nat Med. 2002; 8(7):731-737.
[38] Huang J, Jia Y, Fu T, Viswakarma N, Bai L, Rao M S, Zhu Y, Borensztajn J, Reddy J K. Sustained activation of PPAR{alpha} by endogenous ligands increases hepatic fatty acid oxidation and prevents obesity in ob/ob mice [published online ahead of print Oct. 18, 2011]. FASEB J.
[39] Orellana-Gavalda J M, Herrero L, Malandrino M I, Paneda A, Sol Rodriguez-Pena M, Petry H, Asins G, Van Deventer S, Hegardt F G, Serra D. Molecular therapy for obesity and diabetes based on a long-term increase in hepatic fatty-acid oxidation. Hepatology. 2011; 53(3):821-832.
[40] Bajotto G, Murakami T, Nagasaki M, Qin B, Matsuo Y, Maeda K, Ohashi M, Oshida Y, Sato Y, Shimomura Y. Increased expression of hepatic pyruvate dehydrogenase kinases 2 and 4 in young and middle-aged Otsuka Long-Evans Tokushima Fatty rats: induction by elevated levels of free fatty acids. Metabolism. 2006; 55(3):317-323.
[41] Schummer C M, Werner U, Tennagels N, Schmoll D, Haschke G, Juretschke H P, Patel M S, Gerl M, Kramer W, Herling A W. Dysregulated pyruvate dehydrogenase complex in Zucker diabetic fatty rats. Am J Physiol Endocrinol Metab. 2008; 294(1):E88-96.
[42] Coleman R A, Haynes E B. Hepatic monoacylglycerol acyltransferase. Characterization of an activity associated with the suckling period in rats. J Biol Chem. 1984; 259(14):8934-8938.
[43] Kang H S, Okamoto K, Kim Y S, Takeda Y, Bortner C D, Dang H, Wada T, Xie W, Yang X P, Liao G, Jetten A M. Nuclear orphan receptor TAK1/TR4-deficient mice are protected against obesity-linked inflammation, hepatic steatosis, and insulin resistance. Diabetes. 2011; 60(1):177-188.
[44] Watanabe M, Nakashima H, Ito K, Miyake K, Saito T. Improvement of dyslipidemia in OLETF rats by the prostaglandin I(2) analog beraprost sodium. Prostaglandins Other Lipid Mediat. 2010; 93(1-2):14-19.
[45] Ukawa M, Akita H, Masuda T, Hayashi Y, Konno T, Ishihara K, Harashima H. 2-Methacryloyloxyethyl phosphorylcholine polymer (MPC)-coating improves the transfection activity of GALA-modified lipid nanoparticles by assisting the cellular uptake and intracellular dissociation of plasmid DNA in primary hepatocytes. Biomaterials. 2010; 31(24):6355-6362.
[46] Mudhakir D, Akita H, Khalil I A, Futaki S, Harashima H. Drug Metab Phamacokinet. 20(4):275-81 (2005).
[47] Kakudo T, Chaki S, Futaki S, Nakase I, Akaji K, Kawakami T, Maruyama K, Kamiya H, Harashima H. Biochemistry. 43(19):5618-28 (2004).
[48] Khalil I A, Hayashi Y, Mizuno R, Harashima H. J Control Release. 156(3):374-80 (2011).
[49] Hayashi Y, Yamauchi J, Khalil IA, Kajimoto K, Akita H, Harashima H. Int J Pharm. 419(1-2):308-13 (2011).
[50] Hayashi Y, Mizuno R, Khalil I A, Akita H, Harashima H. (Submitted)
[51] Hayashi Y, Kajimoto K, Sato Y, Afsana A, Suemitsu E, Sakurai Y, Hatakeyama H, Hyodo M, Kaji N, Baba Y, Harashima H. (Submitted)

1. A method for treating and/or preventing type 2 diabetes in a subject, comprising administering to the subject a composition comprising a compound which is able to inhibit expression or activity of monoacylglycerol O-acyltransferase 1 in liver of the subject.

2. The method of claim 1, wherein said compound is dsRNA or antisense targeted against a nucleotide sequence encoding monoacylglycerol O-acyltransferase 1.

3. The method of claim 2, wherein a target sequence of said dsRNA for the mRNA of monoacylglycerol O-acyltransferase 1 is 5′-CCGGGTCACAATTATATATTT-3′ (SEQ ID NO:1).

4. The method of claim 1, wherein said compound is an inhibitor of monoacylglycerol O-acyltransferase 1 activity.

5. The method of claim 4, wherein said inhibitor is antibody or aptamer of monoacylglycerol O-acyltransferase 1.

6. The method of claim 1, wherein said composition comprises said compound in a lipid nanoparticle formulation.

7. The method of claim 6, wherein the lipid nanoparticles are modified with octaarginine and/or GALA.

8. The method of claim 1, wherein said composition does not inhibit monoacylglycerol O-acyltransferase 1 in at least one diabetes related organ other than liver.

9. The method of claim 8, wherein said at least one diabetes related organ other than liver is skeletal muscle, intestine or adipose tissue.

10. The method of claim 8, wherein said diabetes related organs other than liver include skeletal muscle, intestine and adipose tissue.

11. The method of claim 1, wherein the subject is mammal.

12. The method of claim 11, wherein the subject is human.

13. The method of claim 1, wherein the composition is administered into the bloodstream.

14. A composition comprising a compound which inhibits expression or activity of monoacylglycerol O-acyltransferase 1 in liver of a subject formulated for use in the treatment or prevention of type 2 diabetes.

15. The composition of claim 14, wherein said compound is dsRNA or antisense DNA or RNA targeted against a nucleotide sequence encoding monoacylglycerol O-acyltransferase 1.

16. The composition of claim 15, wherein a target sequence of said dsRNA for the mRNA of monoacylglycerol O-acyltransferase 1 is 5′-CCGGGTCACAATTATATATTT-3′ (SEQ ID NO:1).

17. The composition of claim 14, wherein said compound is an inhibitor of monoacylglycerol O-acyltransferase 1 activity.

18. The composition of claim 19, wherein said inhibitor is an antibody or aptamer of monoacylglycerol O-acyltransferase 1.

19. The composition of claim 14, wherein said composition comprises said compound in a lipid nanoparticle formulation.

20. The composition of claim 19, wherein the lipid nanoparticles are modified with octaarginine and/or GALA.

21. The composition of claim 14, wherein said composition does not inhibit monoacylglycerol O-acyltransferase 1 in at least one diabetes related organ other than liver.

22. The composition of claim 21, wherein said at least one diabetes related organ other than liver is skeletal muscle, intestine or adipose tissue.

23. The composition of claim 21, wherein said diabetes related organs other than liver include skeletal muscle, intestine and adipose tissue.

24-26. (canceled)

27. A method to identify a diabetes therapeutic agent or preventive agent, comprising:
(a) contacting a candidate compound with cells in the presence of labeled fatty acid CoA and monoacylglycerol;
(b) extracting lipids from the cells;
(c) determining the amount of diacylglycerol and triacylglycerol in said lipid extract; and
(d) wherein an amount of diacylglycerol and triacylglycerol in said extract lower than in extracts of control cells with which the candidate compound has not been contacted, identifies the candidate compound as a diabetes therapeutic agent or preventive agent.

28. The method of claim 27, wherein the cells are cells which stably over express human monoacylglycerol O-acyltransferase 1.

29. A method of screening a diabetes therapeutic agent or preventive agent, comprising:
(a) contacting a candidate compound with a hepatic parenchymal cell culture in the presence of monoacylglycerol;
(b) detecting an amount of lipid droplets formed in the cells;
(c) wherein an amount of lipid droplets formed in said cells lower than in control cells with which the candidate compound has not been contacted, identifies the candidate compound as a diabetes therapeutic agent or preventive agent.

30. The method of claim 29, wherein the cells are cells which stably over express human monoacylglycerol O-acyltransferase 1.

31. A method of screening a diabetes therapeutic agent or preventive agent, comprising:
(a) administering a candidate compound to a diabetes model animal;
(b) extracting liver from the animal;
(c) detecting an amount of lipid droplets formed in the liver cells;
(d) wherein an amount of lipid droplets formed in said cells lower than in a control model animal to which the candidate compound has not been administered, identifies the candidate compound as a diabetes therapeutic agent or preventive agent.

32. A method of screening a diabetes therapeutic agent or preventive agent, comprising:
(a) administering a candidate compound to a diabetes model animal;
(b) subjecting the animal to at least one test selected from a glucose-tolerance test, an insulin-tolerance test and a pyruvic tolerance test; and
(c) wherein any one following results identifies, the candidate compound as a diabetes therapeutic agent or preventive agent:
in the glucose-tolerance test, a glucose peak level lower than in a control model animal to which the candidate compound has not been administered;
in the insulin-tolerance test, an improved insulin response compared with a control model animal to which the candidate compound has not been administered; and
in the pyruvic tolerance test, reduced de novo hepatic glucose production compared with a control model animal to which the candidate compound has not been administered.
